(19) United States
(12) Patent Application Publication
(10) Pub. No.: US 2025/0259749 A1
(43) Pub. Date: Aug. 14, 2025

(54) HEALTH-JOURNEY BASED COMPUTER AUTOMATED PATIENTS' HEALTH RISKS STRATIFICATION AND INTERVENTIONS

(71) Applicant: FeelBetter Ltd., Giv'atayim (IL)

(72) Inventors: Yoram HORDAN, Herzeliya (IL); Liat PRIMOR-MAROM, Giv'atayim (IL)

(21) Appl. No.: 18/856,730

(22) PCT Filed: Apr. 4, 2023
(86) PCT No.: PCT/IL2023/050366

§ 371 Date: Oct. 14, 2024

(30) Foreign Application Priority Data
Apr. 14, 2022 (IL) 292291

(51) Int. Cl. G16H 50/30; G16H 10/60; G16H 20/10; G16H 40/20; G16H 50/70; G16H 70/40

(52) U.S. Cl. CPC G16H 50/30; G16H 10/60; G16H 20/10; G16H 40/20; G16H 50/70; G16H 70/40

(57) ABSTRACT

Some embodiments relate to computerized methods and systems dedicated to automatic health risks stratification of large population of patients for determining a respective health risk of the patients in the population, which enables to identify patients that exhibit high risk of a medical-care event (e.g., hospitalization risk) and provide urgent interventions to avoid or reduce the likelihood of the occurrence of such an event.

### CROSS-REFERENCE TO RELATED APPLICATIONS

This application is a national phase filing under 35 C.F.R. § 371 of and claims priority to PCT Patent Application No. PCT/IL2023/050366, filed on Apr. 4, 2023, which claims the priority benefit under 35 U.S.C. § 119 of Israeli Patent Application No. 292291, filed on Apr. 14, 2022, the contents of which are hereby incorporated in their entireties by reference.

### FIELD OF THE PRESENTLY DISCLOSED SUBJECT MATTER

The presently disclosed subject matter relates to a computer system and method for automatic monitoring and identification of risks in a patient's medication regimen.

### BACKGROUND

A medication associated risk or error is a failure in the treatment process of a patient and more specifically in the drug administration regimen assigned to a patient. Examples of medication risks and errors include, erroneous prescription of drugs that may include the administration of an inappropriate drug and/or dosage, the administration of an ineffective drug, over prescribing, under prescribing, incorrect frequency or duration of drug administration, adverse drug reactions associated with a drug, drug-drug interactions, etc.

Medication associated risks and errors are very often harmful (or have the potential of being harmful) to patients and may directly induce adverse medical conditions or otherwise exacerbate existing medical conditions. Therefore, medication associated risks and errors have a general negative effect on a patient's health and increase morbidity rates. In addition, medication related risks and errors cause a significant increase in medical-care events, including hospitalization and emergency room (ER) admissions of patients, which cause inconvenience and suffering to patients and incur tremendous expenses for patients, as well as private and public health institutions.

### General Description

While the task of identifying medication associated risks and errors in any medication regimen is challenging, due to the numerous interrelated factors that may have harmful effects on the patient, this task may become considerably more challenging in the case of complex patients such as those that exhibit comorbidity and polypharmacy. Comorbidity is a situation where a single individual suffers from multiple health problems (health conditions), and as a result, is under a complex medication regimen (polypharmacy) involving the use of multiple drugs. One non-limiting example may include an individual suffering from 3 chronic conditions while being administered with a medication regimen that includes 5 drugs or more. Consumption of multiple drugs has been known to affect the safety and effectiveness of the drugs, increase the frequency of drug-to-drug interaction, and, in general, pose a greater risk of causing harm to the patient.

The presently disclosed subject matter includes a computer implemented systems and methods for patients' health risks stratification (classification) and interventions. The disclosed systems and methods are designed for monitoring a patient's medication regimen and automatically identifying medication risks. The disclosed systems and methods are further designed for automatically generating recommendations for changes (also referred to as “interventions”) in the medication regimen, dedicated to improving the medication regimen and avoid or at least reduce potential harm to the patient or to suffer from medication-related complications. This includes the identification of medical-care events (e.g., hospitalization) risk of patients and the provision of interventions which are aimed at changing the medication regimen for reducing the medical-care events risk.

The presently disclosed subject matter further includes computerized methods and systems dedicated to automatic health risks stratification of large population of patients for determining a respective health risk of the patients in the population, which enables to identify patients that exhibit high risk of a medical-care event (e.g., hospitalization risk) and provide urgent interventions to avoid or reduce the likelihood of the occurrence of such an event.

According to a first aspect of the presently disclosed subject matter there is provided a computer implemented method of automatic stratification of a group of patients according to risk of occurrence of a medical-care event, the method comprising:
for each patient, in the group of patients:
processing a patient's health-journey that comprises historical personal medical data of the patient collected over a past period and generating a risks-maps-sequence comprising a plurality of risks-maps, each risks-map in the risks-maps-sequence is generated based on personal medical data which was available at a certain time point along the past period;
wherein the risks-maps-sequence comprises a current risks-map; wherein each risks-map is a data-structure comprising: i) a plurality of relevant health-items, each relevant health-item is a data object that represents a respective health condition which is identified as relevant to a medical status of the patient by at least one activator that includes medical data; and ii) at least one intervention that includes data that prescribes a change to a drug or a treatment that has been identified in the risks-map to be related to a respective health-risk to the patient;
providing the risks-maps-sequence as input to one or more Machine-Learning (ML) models, dedicated to determining a plurality of risk-scores for the current risks-map, that include:
a respective validity score to one or more activators of a relevant health-item in the current risks-map, which indicate collectively a level of certainty that the respective health conditions is relevant to the patient;
a respective severity score to each relevant health-item in the current risks-map, which indicates a severity of a health risk that is related to the respective health condition; and a respective intervention score to each intervention in the current risks-map, that indicates a correlation between the intervention and occurrence of a medical care event; determining, based on the plurality of risk-scores assigned to the current risks-map, a patient's risk-score, that indicates a relative risk of occurrence of a medical-care event to the patient; and stratifying patients in the group of patients according to their respective patient's risk-score.

In addition to the above features, the computer implemented method according to this aspect of the presently disclosed subject matter can optionally comprise in some examples one or more of features (i) to (xii) below, in any technically possible combination or permutation:
i, wherein the intervention is related to a respective relevant health-item in the current risks-map, which represents a health condition that is related to the respective health-risk to the patient, the method further comprising, determining for each relevant health-item in the current risks-map, based on the plurality of risk-scores, a respective health-item risk-score and determining the patient's risk-score based on a combination of the plurality of risk-scores in the current risks-map.
ii. The method further comprising: for each patient in the group:
   providing the risks-maps-sequence as input to a Machine Learning (ML) model dedicated to determining a respective progressive risk-score of the patient that indicates a correlation between a combination of one or more health-item sequences found in the risks-maps-sequence and a risk of occurrence of a medical-care event to the patient;
   determining a combined patient's risk-score based on the patient's risk-score and the progressive risk-score; and stratifying patients in the group of patients according to their respective combined risk-scores.
iii. The method further comprising,
   during generating the current risks-map for each patient:
   processing the patient's health-journey and identifying at least one dynamic activator; wherein a dynamic activator comprises a sequence of medical data values of a certain type recorded in the patient's health-journey, wherein each medical data value in the sequence is recorded at a different time along a period of the health-journey and the sequence of medical data values is characterized by a distinctive pattern; determining a certain health condition that exists in correlation with the dynamic activator; and classifying a health-item representing the certain health condition as a relevant health-item based on the correlation.
iv. The method further comprising:
   identifying in the patient's health-journey, additional medical data (e.g., patient's behavioral data), other than the dynamic activator; determining a health condition that exists in correlation with the dynamic activator and the additional medical data; and classifying a health-item representing the certain health condition as a relevant health-item based on the correlation and the additional medical data.
v. Wherein a plurality of dynamic activators that comprise the same type of medical data are characterized each by a different distinctive pattern, and is each correlated with a different health condition of a plurality of health conditions; the method further comprising:
   determining respective features charactering the distinctive pattern;
   determining based on the respective features a correlation between the dynamic activator and a specific health condition out of the plurality of health conditions; and identifying a health-item representing the specific health condition as a relevant health-item.
vi. The method further comprising determining the at least one intervention comprising: identifying in the relevant health-items, one or more risk-related health-items that are each indicative of a health risk related to a drug prescribed to the patient, comprising:
   obtaining from the personal medical data of the patient, information about administration of a drug or treatment for treating a respective health condition; comparing the information with data indicating recommended administration of the drug or treatment; classifying the respective relevant health-item representing the health condition as risk-related in case a discrepancy is found between the information and the recommended administration; and generating an intervention dedicated to correcting the discrepancy.
vii. The method further comprising determining the at least one intervention comprising:
   identifying in the relevant health-items, one or more risk-related health-items that are each indicative of a health risk related to a drug prescribed to the patient, comprising:
   obtaining from the personal medical data of the patient, information about a drug that is being administered for treating a health condition and that is related to a health risk which may be caused from usage of the drug; and generating an intervention dedicated to alleviating the health risk.
viii. Wherein the medical-care event includes any one of hospitalization and ER admission.
ix. The method further comprising determining the at least one intervention comprising:
   determining for each relevant health-item in each risks-map in the risks-maps-sequence a respective health-item risk-score; identifying one or more changes in a drug or treatment in a medication regimen observed along the patient's health-journey; identifying one or more changes in health-item risk-scores of respective relevant health-items observed along the risks-maps-sequence; identifying a correlation between a time of a change in a drug or treatment and a time of a change in a health-item risk-score of a respective relevant health-item; deducing based on the correlation that the change in the drug or treatment caused the change of the health-item risk-score of the respective relevant health-item; generating, according to the deducing, an intervention dedicated to alleviating a health risk related to the respective drug or treatment.
x. Wherein the at least one change in the respective drug or treatment is commencement of taking a new drug, the method further comprising obtaining data indicative of a respective drug delay onset period and adapting the time of the change according to the respective drug delay onset period.
xi. Wherein the one or more ML models includes:
   a first ML model dedicated to assigning validity scores based on combinations of validity features identified in the risks-maps-sequence;
   a second ML model dedicated to assigning severity scores based on combinations of severity features identified in the risks-maps-sequence; and
   a third ML model dedicated to assigning interventions scores based on combinations of interventions features identified in the risks-maps-sequence.
xii. Wherein the current risks-map is generated based on most updated personal medical data in the patient's health-journey.

The presently disclosed subject matter further contemplates a computer system comprising a processing circuitry that comprises at least one processor and a computer memory, the processing circuitry is configured to execute a method as described above with reference the first aspect and may optionally further comprise one or more of the features (i) to (xii) listed above, mutatis mutandis, in any technically possible combination or permutation.

The presently disclosed subject matter further contemplates a non-transitory computer readable storage medium tangibly embodying a program of instructions that, when executed by a computer, cause the computer to perform a method as described above with reference the first aspect, and may optionally further comprise one or more of the features (i) to (xii) listed above, mutatis mutandis, in any technically possible combination or permutation.

The presently disclosed subject matter further contemplates a patients' health risk stratification computer-product operable in a computer and recorded on a non-transitory computer-readable medium for automatic stratification of a group of patients according to risk of occurrence of a medical-care event, wherein the product is produced by the processes that includes steps as described above with reference the first aspect and may optionally further comprise one or more of the features (i) to (xii) listed above, mutatis mutandis, in any technically possible combination or permutation.

According to a second aspect of the presently disclosed subject matter there is provided a computer implemented method of training a machine leaning model dedicated to assigning risk-scores for a risks-map, to be used collectively to determine patients' risks-score to enable stratification of patients according to a respective risk of occurrence of a medical-care event, wherein the risks-map comprises: a plurality of relevant health-items, each relevant health-item is a data object that represents a respective health condition which is identified as relevant to a medical status of the patient by at least one activator that includes medical data; and at least one intervention that prescribes a change to a drug or a treatment that has been identified in the risks-map to be related to a health-risk to the patient;
the method comprising:
obtaining a plurality of health-journeys, each health-journey comprises historical personal medical data of a respective patient collected over a past period;
generating from each health-journey a respective risks-maps-sequence, thereby obtaining a plurality of risks-maps-sequence, each comprising a plurality of risks-maps, each risks-map in the risks-maps-sequence is generated based on personal medical data which was available at a certain time point along the past period; wherein the each risks-maps-sequence further comprising information about recorded medical-care events; wherein each risks-maps-sequence comprises data about medical-care events recorded for the respective patient;
using the plurality of risks-maps-sequences as a training dataset for training one or more machine-learning (ML) models, to provide risk-scores that are indicative of a correlation between features extracted from the risks-maps-sequences and a risk of occurrence of medical-care event.

Wherein in some examples the features include, interventions features, and wherein the one or more machine-learning (ML) models include a first ML model, that is made capable, following training, to assign intervention scores to intervention in the risks-map.

Wherein in some examples the features include, validity features, and wherein the one or more machine-learning (ML) models include a second ML model, that is made capable, following training, to assign validity scores to activators in the risks-map.

Wherein in some examples the features include, severity features, and wherein the one or more machine-learning (ML) models include a third ML model, that is made capable, following training, to assign severity scores to health-items in the risks-map.

The presently disclosed subject matter further contemplates a computer system comprising a processing circuitry that comprises at least one processor and a computer memory, the processing circuitry is configured to execute a method as described above with reference the second aspect.

The presently disclosed subject matter further contemplates a non-transitory computer readable storage medium tangibly embodying a program of instructions that, when executed by a computer, cause the computer to perform a method as described above with reference the second aspect.

According to a third aspect of the presently disclosed subject matter there is provided a computer implemented method of automatic stratification of health risks in a medication regimen administered to a patient, the method comprising:
generating a risks-map data-structure comprising:
selectively adding to the data-structure a plurality of relevant health-items, each relevant health-item is a data object that represents a respective health condition which is identified as relevant to a medical status of the patient by at least one activator that includes medical data;
processing a patient's health-journey that comprises historical personal medical data of the patient collected over a past period and identifying at least one dynamic activator; wherein a dynamic activator comprises a sequence of medical data values of a certain type recorded in the patient's health-journey, wherein each medical data value in the sequence is recorded at a different time along a period of the health-journey and the sequence of medical data values is characterized by a distinctive pattern;
determining a certain health condition that exists in correlation with the dynamic activator; and
classifying a health-item representing the certain health condition as a relevant health-item based on the correlation.

In addition to the above features, the computer implemented method according to this aspect of the presently disclosed subject matter can optionally comprise one or more of features (i) to (iii) below, in any technically possible combination or permutation:
i. The method further comprising:
   identifying in the patient's health-journey, additional medical data, other than the dynamic activator; determining a health condition that exists in correlation with the dynamic activator and the additional medical data; and classifying a health-item representing the certain health condition as a relevant health-item based on the correlation and the additional medical data.
ii, wherein a plurality of dynamic activators that comprise the same type of medical data are characterized each by a different distinctive pattern, and is each correlated with a different health condition of a plurality of health conditions; the method further comprising:
   determining respective features charactering the distinctive pattern;
   determining based on the respective features a correlation between the dynamic activator and a specific health condition out of the plurality of health conditions; and identifying a health-item representing the specific health condition as a relevant health-item.
iii. The method further comprising:
   determining at least one relevant health-item in the risks-map that is indicative of a health risk related to a drug or treatment prescribed to the patient and at least one respective intervention that prescribes a change to the drug or a treatment dedicated to alleviating the health risk; determining the at least one respective intervention comprising:
   processing the patient's health-journey and generating a respective risks-maps-sequence comprising a plurality of risks-maps, each risks-map in the risks-maps-sequence is generated based on personal medical data which was available at a certain time point along the past period; determining for each relevant health-item in each risks-map in the risks-maps-sequence a respective health-item risk-score; generating at least one intervention that prescribes a change to a drug or a treatment that has been identified in the risks-map to be related to a health-risk to the patient, comprising:
   identifying one or more changes in a respective drug or treatment in the medication regimen observed along the patient's health-journey; identifying one or more changes in health-item risk-scores of respective relevant health-items observed along the risks-maps-sequence; identifying a correlation between a time of at least one change in a respective drug or treatment and a time of at least one change in health-item risk-score; deducing based on the correlation that the at least one change in the drug or treatment caused the change of the health-item risk-score of the at least one relevant health-item; generating, according to the deducing, the at least one respective intervention dedicated to alleviating a health risk related to the respective drug or treatment.

The presently disclosed subject matter further contemplates a computer system comprising a processing circuitry that comprises at least one processor and a computer memory, the processing circuitry is configured to execute a method as described above with reference the third aspect and may optionally further comprise one or more of the features (i) to (iii) listed above, mutatis mutandis, in any technically possible combination or permutation.

The presently disclosed subject matter further contemplates a non-transitory computer readable storage medium tangibly embodying a program of instructions that, when executed by a computer, cause the computer to perform a method as described above with reference the third aspect, and may optionally further comprise one or more of the features (i) to (iii) listed above, mutatis mutandis, in any technically possible combination or permutation.

According to a fourth aspect of the presently disclosed subject matter there is provided a computer implemented method of automatic stratification of health risks in a medication regimen administered to a patient, the method comprising:
generating a risks-map data-structure comprising:
selectively adding to the data-structure a plurality of relevant health-items, each relevant health-item is a data object that represents a respective health condition which is identified as relevant to a medical status of the patient by at least one activator that includes medical data;
wherein the risks-map is a data-structure comprising: i) a plurality of relevant health-items, each relevant health-item is a data object that represents a respective health condition which is identified as relevant to a medical status of the patient by at least one activator that includes medical data; and ii) at least one intervention that includes data that prescribes a change to a drug or a treatment that has been identified in the risks-map to be related to a respective health-risk to the patient;
determining the at least one intervention comprising:
processing a patient's health-journey that comprises historical personal medical data of the patient collected over a past period and generating a respective risks-maps-sequence comprising a plurality of risks-maps, each risks-map in the risks-maps-sequence is generated based on personal medical data which was available at a certain time point along the past period;
determining for each relevant health-item in each risks-map in the risks-maps-sequence a respective health-item risk-score;
generating at least one intervention that prescribes a change to a drug or a treatment that has been identified in the risks-map to be related to a health-risk to the patient, comprising:
identifying one or more changes in a respective drug or treatment in the medication regimen observed along the patient's health-journey;
identifying one or more changes in health-item risk-scores of respective relevant health-items observed along the risks-maps-sequence;
identifying a correlation between a time of a change in a respective drug or treatment and a time of a change in health-item risk-score of a respective relevant health-item;
deducing based on the correlation that the change in the drug or treatment caused the change of the health-item risk-score of the respective relevant health-item;
generating the at least one respective intervention dedicated to alleviating a health risk related to the respective drug or treatment.

The presently disclosed subject matter further contemplates a computer system comprising a processing circuitry that comprising at least one processer and computer memory, the processing circuitry is configured to execute a method as described with respect to the fourth aspect.

The presently disclosed subject matter further contemplates a computer product operable in a computer and recorded on a non-transitory computer-readable medium for automatic stratification of health risks in a medication regimen administered to a patient, wherein the product is produced by the processes as described with respect to the fourth aspect.

The presently disclosed subject matter further contemplates a non-transitory computer readable storage medium tangibly embodying a program of instructions that, when executed by a computer, cause the computer to perform a method as described with respect to the fourth aspect.

### BRIEF DESCRIPTION OF THE DRAWINGS

In order to understand the presently disclosed subject matter and to see how it may be carried out in practice, the subject matter will now be described, by way of non-limiting examples only, with reference to the accompanying drawings, in which:

FIG. 1 is high level schematic illustration of a system, according to some examples of the presently disclosed subject matter;

FIG. 2 is a block diagram schematically illustrating a more detailed view of the system, according to some examples of the presently disclosed subject matter;

FIG. 3 is a flowchart showing operations performed as part of risk stratification and intervention process, according to some examples of the presently disclosed subject matter;

FIG. 4 is a flowchart showing operations carried out as part of a risks-map generation process, according to some examples of the presently disclosed subject matter;

FIG. 5 it a schematic illustration of data generated as part of the personalized risks-map, according to some examples of the presently disclosed subject matter;

FIG. 6 is a schematic illustration of a personalized risks-map, according to some examples of the presently disclosed subject matter;

FIG. 7 is an example of graphical representation of some components of a health-journey, according to some examples of the presently disclosed subject matter;

FIG. 8 is a block diagram schematically illustrating a more detailed view of components of the health-journey engine 140, according to some examples of the presently disclosed subject matter;

FIG. 9 is a flowchart showing operations carried out for determining dynamic activators, according to some examples of the presently disclosed subject matter;

FIG. 10 is flowchart showing operations carried out for determining ML-enhanced core risk-scores, according to some examples of the presently disclosed subject matter;

FIG. 11 is a flowchart showing operations carried out for determining progressive patients' risk-scores, according to some examples of the presently disclosed subject matter; and

FIG. 12 is a flowchart showing operations carried out for generating interventions using a risks-maps-sequence, according to some examples of the presently discloses subject matter.

### DETAILED DESCRIPTION

In the drawings and descriptions set forth, identical reference numerals indicate those components that are common to different embodiments or configurations. Elements in the drawings are not necessarily drawn to scale.

Unless specifically stated otherwise, as apparent from the following discussions, it is appreciated that throughout the specification discussions utilizing terms such as “obtaining”, “determining”, “generating”, “comparing”, “adding” or the like, include an action and/or processes of a computer that manipulate and/or transform data into other data, said data represented as physical quantities, e.g., such as electronic quantities, and/or said data representing the physical objects.

The terms computer/computer device/computerized system, or the like, should be expansively construed to include any kind of hardware-based electronic device with a processing circuitry (e.g., digital signal processor (DSP), a GPU, a TPU, a field programmable gate array (FPGA), an application specific integrated circuit (ASIC), microcontroller, microprocessor etc.). The processing circuitry can comprise for example, one or more processors operatively connected to computer memory, loaded with executable instructions for executing operations as further described below.

The terms “client” and “server” as used herein below may include, but are not limited to, computers, server computers, personal computers, portable computers, Smartphones, appliances, watches, cars, televisions, voice-controlled assistants, tablet devices, or any other hardware computerized device configured with adequate processing and communication resources.

The operations in accordance with the teachings herein may be performed by a computer specially constructed for the desired purposes, or by a general-purpose computer specially configured for the desired purpose by a computer program stored in a computer readable storage medium.

As used herein, the phrase “for example,” “such as”, “for instance” and variants thereof, describe non-limiting embodiments of the presently disclosed subject matter. Reference in the specification to “one case”, “some cases”, “other cases”, or variants thereof, means that a particular feature, structure or characteristic described in connection with the embodiment(s) is included in at least one embodiment of the presently disclosed subject matter. Thus, the appearance of the phrase “one case”, “some cases”, “other cases” or variants thereof does not necessarily refer to the same embodiment(s).

It is appreciated that certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

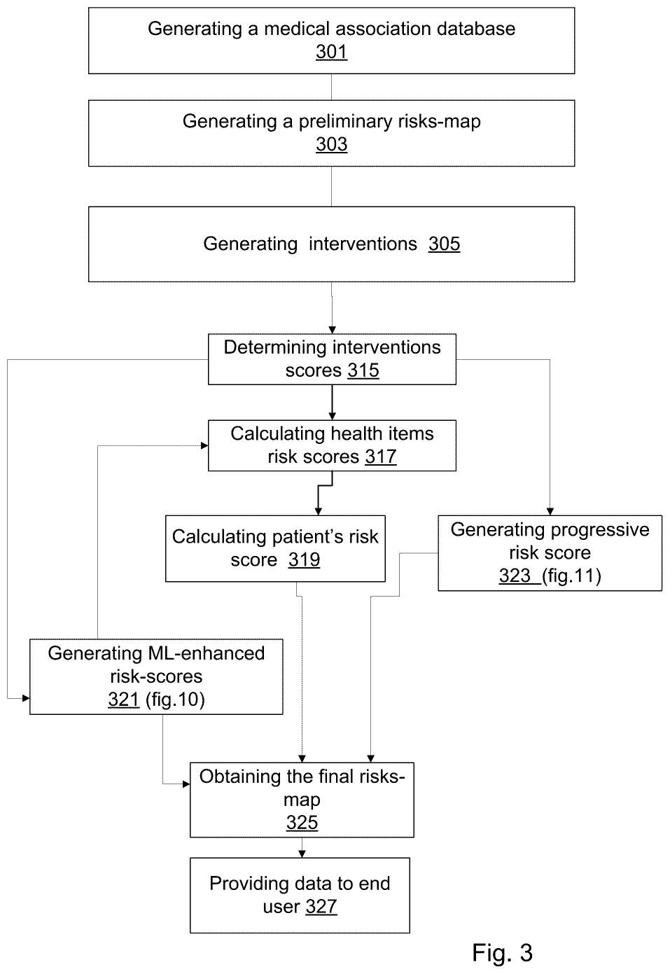

In embodiments of the presently disclosed subject matter, fewer, more and/or different stages than those shown in FIGS. 3, 4, and 9 to 12 may be executed. In embodiments of the presently disclosed subject matter, one or more stages illustrated in FIGS. 3, 4, and 9 to 12 may be executed in a different order and/or one or more groups of stages may be executed simultaneously. For example, operations described with reference to blocks 417 and 419 may be executed simultaneously or in reverse order.

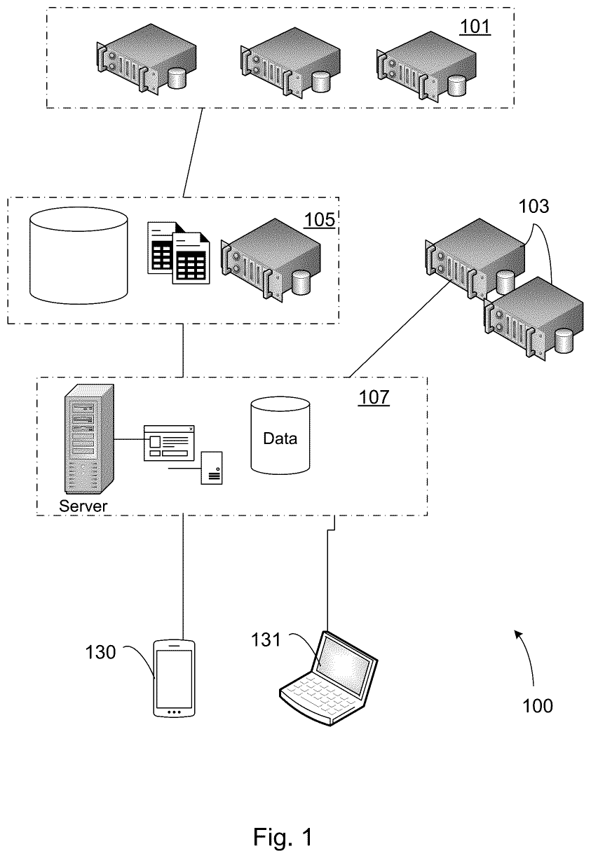

FIGS. 1, 2 and 8 schematically illustrate various components of the system architecture in accordance with certain examples of the presently disclosed subject matter. Elements in FIGS. 1, 2 and 8 can be made up of a combination of software and hardware and/or firmware that performs the functions as defined and explained herein. Elements in FIGS. 1, 2 and 8 may be centralized in one location or dispersed over more than one location. For example, MSIS server 107 can be distributed over a plurality of computer devices, each possibly located at a different geographical location, or can be otherwise centralized in a single computer device. In other embodiments of the presently disclosed subject matter, the system may comprise fewer, more, and/or different elements than those shown in FIGS. 1, 2 and 8. It is noted that the specific division of the functionally of the disclosed system into specific parts as described below, is provided by way of example, and other alternatives are also construed within the scope of the presently disclosed subject matter.

Bearing the above in mind, attention is now drawn to FIG. 1 which shows a high-level view of a system, according to examples of the presently disclosed subject matter. The network architecture in FIG. 1 is a general example which demonstrates some principles of the presently disclosed subject matter and may vary in structure and therefore should not be construed as limiting. System 100 may be implemented over any type of communication network. For example, communication can be realized over any one of the following networks: the Internet, a local area network (LAN), wide area network (WAN), metropolitan area network (MAN), any type of telephone network (including for example PSTN with DSL technology) or mobile network (Including for example 3G, 4G or 5G mobile communication technologies), or any combination thereof.

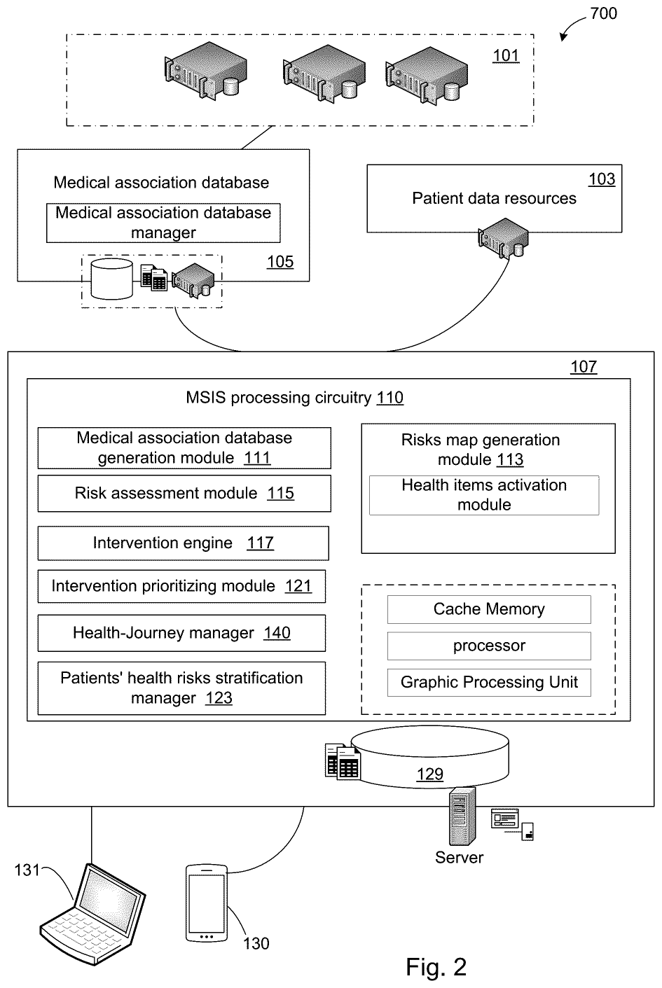

System 100 is configured in general to provide a medical risk-stratification and intervention service (referred to herein in general as “medication stratification and intervention service” or “MSIS” in short). As shown, a user (e.g., patient and/or medical practitioner) can use a client device (130/131) to communicate and interact with an MSIS server 107 to obtain the medication intervention service, as further explained below. Notably, multiple client devices can interact with the system simultaneously. Examples of specific functional elements of MSIS server 107 are shown in FIG. 2 and are described below. As shown in FIG. 2, according to some examples, MSIS server device 107 comprises MSIS processing circuitry 110 configured to execute several functional modules in accordance with computer-readable instructions implemented on a non-transitory computer-readable storage medium. Such functional modules are referred to hereinafter as comprised in the processing circuitry. In various examples, processing circuitry 110 comprises or is otherwise operatively connected to various components such as one or more computer processors and memory devices (e.g., cache memory).

According to some examples, MSIS server 107 comprises or is otherwise connected over a communication network to a medical database 105 (otherwise referred to as “medical association database”), configured to store data linking between various medical conditions (otherwise referred to as “health conditions”) and related medical data. The related medical data includes clinical and pharmacological information such as:

Medications and other treatments which are used for treating the respective medical condition, data indicating recommended usage or administration of the medication or treatment (e.g. dosage, frequency, duration, time of day, etc.), adverse drug reactions or symptoms that could be exhibited in patients that use these medications or treatment, and other warnings related to drug usage, such as mixture of different medications (DDI-drug to drug interaction) or warnings related to age groups or other affinity groups. Medical database 105 can be configured to obtain the medical data from to one or more medication data resources 101 as further explained below.

In some examples, MSIS server 107 is further connected to a patients' data resources 103 which can provide medical information on patients. Patients' data resources include for example electronic medical records (EMR) data-repositories (e.g., stored at various computer storage devices accessible by respective servers), that enable to obtain the EMR of a specific patient and/or claims data that relate to a specific patient.

Turning now to FIG. 3, this is a flowchart of operations performed by system 100 (and more specifically by processing circuitry 110) during the risk stratification and intervention process, according to some examples of the presently disclosed subject matter. It is noted that while the operations described with reference to FIG. 3, as well as other figures, refers to a single patient, the presently disclosed subject matter contemplates the application of these operations on large populations (e.g., tenths of thousands to millions) of patients concurrently as further described below. The medical status of a patient (including administered medical treatments such as drugs and other treatments, hereinafter “medication regimen”) currently being provided to the patient are analyzed in order to validate proper medication regimens, determine whether they include any medication errors and/or medication risks, and whether any optimization can be applied on the medical regimen in order to improve its efficiency in treating the relevant medical conditions and/or improve the patients wellbeing (e.g., reduce adverse drug effects) and/or reduce potential risk of occurrence of a medical-care event.

In general, medication errors include any treatment in a medication regimen that fails to achieve its intended use and/or cause (or has the potential to cause) harm to the patient. In case a medication error is identified, recommendations for changes to the medication regimen (otherwise referred to herein as “interventions”), directed to remedy such errors and/or reduce related risks, are provided.

The medical risk-stratification and intervention process of a patient's medication regimen, as disclosed herein, can be performed as a recurring process, performed routinely, e.g., according to a specific scheduling scheme for the purpose of providing continuous evaluation and improvement to the patient's medication regimen. In another example, the process can be executed when a need arises to approve a change in the patient's medication regimen, e.g., a new drug is added to the patient's medication regimen.

For example, consider a patient submitting a request for renewal of a subscription to a certain drug. As part of the process of approving renewal of the subscription, a medical risk-stratification and intervention process can be executed, that automatically checks for any changes in the medical status of the patient that may have occurred since the last renewal, and whether these changes, if occurred, influence the risk related to the requested drug. Furthermore, as part of assessment of the patient's medication regimen, an analysis of the patient's trends over time and linkage to different clinical or pharmacological changes can be incorporated to better identify possible risks and causes and provide recommended interventions, accordingly.

It is noted that although the generation of the medical database is described as part of the operation flow in FIG. 3, this is done for simplicity and ease of understanding only and may be otherwise executed as a separate process (e.g., executed asynchronously by a different computer device), where the generated databased is used for the generation of the personalized risks-map. It is further noted that generation of the medical database as disclosed with reference to block 301 may not be repeated each time the process is executed. Rather, generation of the medical database can be done once and then updated from time to time asynchronously from the execution of other operations related to the risk-stratification and intervention process.

According to some examples, at block 301 a medical database is generated. The medical database (otherwise referred to as “medication model”) is composed of a collection of individual health-items. Each health-item is a data object that pertains to a specific medical condition or symptom that may inflict patients.

A non-exhaustive list of examples of medical conditions or symptoms that may be assigned with a respective health-item include: hypertension (e.g., indicative of high blood pressure, over a certain threshold); hypotension, headaches; stomach aches; muscle aches; palsy; tremor; insomnia/sleep deprivation; weakness; nausea; vomiting; depression; anxiety; fatigue; confusion; constipation; diarrhea; allergic conjunctivitis; hypoglycemia; hyperglycemia hypokalemia; hypomagnesemia; hyponatremia, cytopenia, and so on.

Each health-item comprises or is otherwise logically associated or linked to various data elements (e.g., data fields or data objects), where different data elements describe medical data related to the medical condition. Medical data in the medical database 105 can include for example the following data elements: appropriate treatments and medication; various indications of the medical condition (such as lab results and/or blood pressure results and the value ranges that are indicative of the medical condition) and/or possible related symptoms; other commonly associated medical conditions; warnings about medical conditions; pharmacologic data such as: recommended drug usage for treating the condition, such as dosing, time of day and frequency indexes; specific dosing indexes related to patients with different impairments (e.g., renal, hepatic); warnings about drug usage and risks according to affinity groups, adverse drug reactions (ADR), drug to drug interaction (DDI), other specific drug warnings, chemical components of drugs, etc.

As mentioned above, according to examples of the presently disclosed subject matter, the relevant medical data can be obtained for example from medical data resources (101), which are generally available to medical staff and researchers and include for example: online medical literature, medical coding and classification systems (e.g. ICD codes of the World Health Organization and CPT codes that are published by the American Medical Association), drug label information provided by the FDA, warning guidance databases, adverse drug reactions (ADR) databases, Drug to Drug interaction (DDI) information, drug duplications databases, medication warnings and guidance (e.g. NNT/NNH, impairments/grouping, comorbidities), etc.

The medical resources 101 are analyzed to obtain the relevant medical data related to each medical condition that is added to the medical database. This includes extraction of various causes, treatments (e.g., drugs) symptoms, indications, and risks, which are related to different heath conditions. In some examples, a deeper analysis is performed on the retrieved data. For example, pharmacological data can be analyzed for the purpose of determining the pharmacological/chemical components of drugs and the effect these specific chemical components may have regarding different medical conditions, risks and/or symptoms, mechanism of action (MoA), etc. One specific example is the analysis of the mechanism of action related to increased risk of falling caused by a specific drug and determining a specific drug component that may be the cause of falling and storing this information in the related health-item.

A specific example of a health-item and associated data is a sleep deprivation health-item that can be associated with data elements related to one or more of: medical conditions that may be indicative of sleep deprivation, specific drugs that may cause sleep deprivation, combinations of two or more drugs (DDI) that may cause sleep deprivation, questionnaire based indexes that may point to sleep deprivation (e.g., Insomnia Severity Index), various medical measurements (e.g., blood pressure, blood test values) that are known to be associated with sleep deprivation, various treatments and drugs that may be administered for treating sleep deprivation, etc.

Another specific example of a health-item and the associated data is a hemophilia health-item that can be associated with data elements related to different factors that may cause/contribute/exacerbate hemophilia, different treatments that are administered for treating hemophilia and/or medical measurements/conditions that may be indicative of hemophilia. This includes drugs that may cause hemophilia, combinations of two or more drugs (DDI) that may cause hemophilia, other medical conditions and symptoms that are known to be associated with hemophilia, medical treatments that may cause or exacerbate hemophilia, various treatments and drugs that may be administered for treating hemophilia, and so on.

The medical database 105 can further include specific information describing the details of each data element. For example, given a certain medical condition and a respective data element corresponding to a drug used for treating the medical condition, the specific details of the recommended drug administration are also provided as part of the database (e.g., various dosage and/or frequency indexes, according to age, weight, severity of symptoms, etc.). In another example, given a certain drug for treating a certain medical condition, the database may also include information about DDIs, warnings, and/or ADRs related to the drug. The information may further include specific complementary conditions that are required to render the warning or ADR effective or more severe. For instance, age (e.g., a warning relevant only to a specific age group such as elderly patients), measurements (a warning relevant if a medical measurement shows values above or below a certain value or within a certain range), specific (additional) medical condition (e.g., a warning is relevant only if the patient is suffering from another specific medical condition), etc.

The abundance of health-items recorded in the medical database 105, which in some examples are defined in the hundreds or more, is intended to provide highly granular data describing specific medical conditions in detail, to thereby enable the representation and analysis of medical regimens of patients with high resolution and precision. It is noted however that the specific assignment of medical conditions to health-items as well as the specific granulation of the health-items may vary and the presently disclosed subject matter is not bound to any specific assignment or granulation.

In some examples, the medical database is generated and managed by a dedicated server. For example, server 105 can comprise a database manager implemented by a processing circuitry configured to parse the relevant data obtained from the various medical data sources 101 and identify within the data, medical data related to various medical conditions. Once the medical data is obtained, medical data related to a certain medical condition, is associated with (e.g., linked to) the respective health-item that represents the medical condition. According to other examples, the medical database is generated by MSIS processing circuitry 110 (e.g., by medical database generation module 111) configured for generating and managing the medical database.

The medical database 105 can be implemented using various techniques, data-structures and forms which are well known in the art. In one example, each entry in the medical database (e.g., a row in a relational database) corresponds to a respective health-item, representing in turn a specific medical condition (or symptom). The fields referenced by each entry can accommodate the medical data extracted from the data resources (101), including the different data elements related to the medical condition as explained above.

Notably, different data elements can be linked to more than one health-item. For example, injected corticosteroids are known to have side effects that include the following medical conditions: stomach irritation, such as indigestion or heartburn, tachycardia (rapid heartbeat), nausea, insomnia, and metallic taste in the mouth. Therefore, each respective health-item representing any one of the above medical conditions, can comprise or otherwise be linked to a data element representing injected corticosteroids, indicating a possible causing agent (or “health-item trigger”).

According to some examples, the medical database 105 further includes information on possible health-item triggers. The term “health-item trigger” (or “trigger” in short) is used herein to include possible indications or causes of certain medical conditions. A trigger is linked in the medical database 105 to the respective health-item representing the medical condition that may be potentially indicated or caused by the trigger.

One example is described above with respect to corticosteroids. Another example is Amitriptyline, which is a medicine used for treating various mental disorders. The list of ADRs related to Amitriptyline includes fatigue, xerostomia, headache, constipation, visual disturbance, dizziness, and weight gain. The list of warnings related to Amitriptyline includes anticholinergic effect, cardiovascular event, conduction disorders, tachycardia, bone marrow suppression, bipolar disorder, and orthostatic hypertension. Thus, Amitriptyline can be considered as a potential trigger of any one of the above medical conditions. In some examples, each of these health-items is associated with specific activation conditions, which must be complied to render Amitriptyline an effective trigger of the health-item.

The triggers can be added to the medical database 105 using various techniques, data-structures and forms which are well known in the art. Triggers can be implemented as data objects like health-items. In some examples, the medical database 105 can be designed with multiple references, such that each health-item may reference one or more respective data elements and each data element may reference one or more health-items. Specifically, data elements that represent triggers of medical conditions are linked to the respective medical condition which they trigger. According to other examples, the medical database may include more than one individual data structure (e.g., data table), where at least one data structure links between the health-items and the respective data elements, and at least one other data structure links between different data elements (health-items triggers, causes and symptoms) and the respective health-items which they trigger.

While all the health-items constituting the medical database 105 represent a full variety of medical conditions that may potentially inflict patients, each patient normally suffers from a subset of the medical conditions in the database.

At block 303 a preliminary personalized risks-map is generated (e.g., by risks-map generation module 113 in MSIS processing circuitry 110) for any specific patient. In general, a personalized risks-map (or “risks-map”) is composed of a collection (subset) of health-item objects selected from the medical database 105, which are relevant to the specific patient (referred to herein below as “relevant health-items”) and provide the specific medical picture (status) of that patient, indicating the specific medical conditions and health risks that are relevant to the patient. The risks-map is considered preliminary since later in the process additional data is added to risks-map to bring it to its final form. A risks-map can be implemented using various techniques, data-structures (e.g., graph) and forms which are well known in the art.

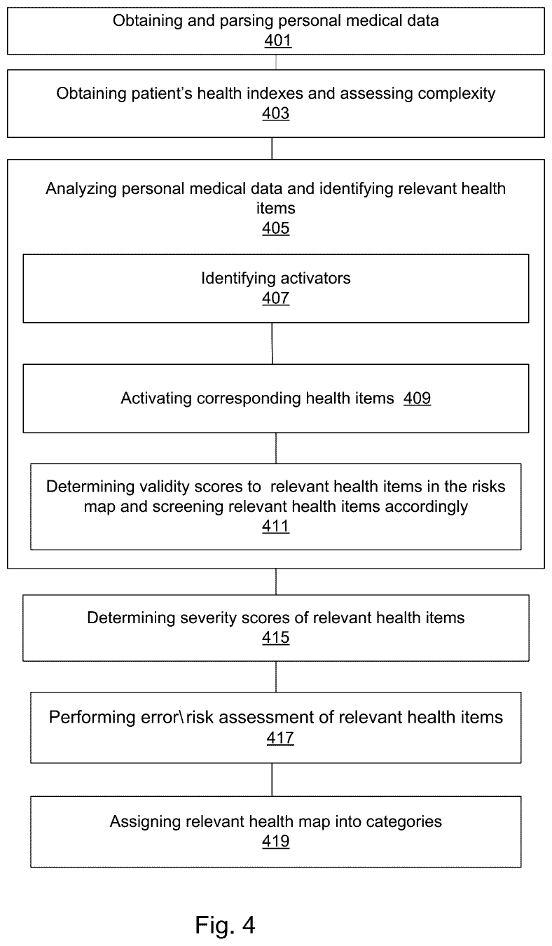

Turning now to FIG. 4, it illustrates a more detailed flowchart of operations carried out during generation of a personalized risks-map, according to some examples of the presently disclosed subject matter. The description reverts to FIG. 3 later below.

At block 401, available personal medical data (including for example, clinical and pharmacological data) of a patient is obtained and analyzed. In some examples, the personal medical data that is retrieved is stored in a dedicated data-repository operatively connected to system 107 (e.g., 129).

Non-limiting examples of patients' personal medical data includes: Patient's electronic medical records (EMR) and claims records including:
i. Personal information such as: patient's age, height, weight, gender, ethnicity and possibly other demographic data and/or social determinants of health;
ii. Clinical information including, but not limited to:
   Medical conditions that the patient is suffering from as well as medical background and history;
   Patient's medical measurements and lab results, including for example, past blood tests and blood pressure results, urine tests, EEG, ECG, medical imaging results such as: X-ray, MRI, ultrasound, PET scan, CT, etc.;
   Patients clinical indexes providing insights to the patient's clinical status (e.g., Patient Health Questionnaire-9 (PHQ9), Mini Nutritional Assessment (MNA), 3IQ, Creatinine Clearance Test (CCT), Child Pugh Score);
   Information about the patient's frailty and ability to perform activities of daily living (e.g., geriatric assessment);
   Various treatments administered to the patient other than drugs such as: dialysis, regular oxygen support, psychological treatment for psychiatric disorders, etc.
iii. Pharmacological information including:
   Patient's medication regimen specifying the prescription drugs being administered to the patient (e.g., by primary care physicians or specialists) as well as the respective indications, dosages, frequencies, etc.; and
   Consumed drugs and vitamins that are bought over the counter or supplements used and which are recorded in the patient's EMR.

According to some examples, in addition to the personal patient's medical information mentioned above, subjective patient data (complementary) is also used by the processing circuitry 110 for generation of a personalized risks-map of a certain patient. The subjective patient data is obtained by direct interaction with the patient for example, using a user interaction platform running on a client device 130 (e.g., smartphone, smart home assistants) and presenting a questionnaire to the patient.

According to some examples, health indexes and the complexity of the patient are assessed (blocks 403). Health indexes may include renal impairment indexes, such as Creatinine Clearance Test, hepatic impairment indexes or other indexes associated with the patient's health. These indexes are used to identify specific risk related factors which are relevant to drug consumption under certain conditions such as renal impairment which influence the medication regimen. To this end, the personal medical data of the patient (e.g., patient's EMR) is analyzed to determine whether it indicates a health index.

As mentioned above, complex patients such as those that exhibit polypharmacy, suffer from multiple health problems and chronic conditions, and are provided with complex medication regimens involving the use of multiple drugs. In some examples, risk assessment of complex patients is different to that of other individuals with low complexity. For example, specific blood measurements associated with the patient's conditions may have different in-range definitions, which depend on the patient's complexity, and accordingly the dosages of medications that are appropriate for these patients may be different to those of the general population. Therefore, patients' complexity is evaluated, and the medical risk assessment of their medication regimen is adapted to restrictions related to their specific complexity. For example, the acceptable dosage range of the medications are adapted according to the indexes and/or complexity.

In some examples, complexity of the patient is evaluated based on the number and type of medical conditions which are identified and/or treatments the patient is receiving regularly. For example, complexity increases with the increase in the number of medical conditions and/or the number of treatments (e.g., dialysis or regular usage of oxygen support).

The personal medical data of a patient is analyzed to identify relevant health-items (block 405; e.g., by health-items activation module in processing circuitry 110). In some examples, the identification of relevant health-items is based on the identification of health-item activators (block 407). The term “health-item activator” is used herein to include personal patient's medical data that may indicate that a certain health-item in the medical database 105 is relevant to a certain patient, thus “lighting-up” or “activating” the health-item and rendering it relevant to the patient's medical risk assessment and adding it as a relevant health-item in the patient's risks-map.

As explained above, health-items in the medical database 105 are associated with one or more data elements corresponding to medical data, including clinical and pharmacological information that is related to the respective medical condition. Accordingly, personal medical data of a patient, including that which is extracted from the patient's EMR, is compared with medical data in the medical database 105 (referred to herein also as “general medical data”) and health-items found to be related to the personal medical data are activated (identified as relevant health-items) and added to the collection of health-items which constitute the patient's personalized risks-map. In other words, the medical database indicates the connection between medical data and respective medical conditions, if the personal medical data of the patient is found to include data that is related to a certain medical condition, the respective health-item representing the medical condition can be activated. Different medical conditions may be related to different medical data, and therefore respective health-items are activated by the specific personal medical data to which it is related.

According to some examples, a first type of health-item activators is “explicit health-item activators”. This type of activators has a straightforward and explicit relationship with health-items. An example of an explicit activator is a medical (clinical) condition reported in the patient's EMR (also referred to herein as “reported medical condition”) that indicates the relevance of a corresponding health-item in the association database that represents the medical condition. For each medical condition disclosed in the personal medical data of the patient, at least one health-item representing the medical condition is activated-added to the patient's personalized risks-map. For example, if the EMR indicates that the patient is suffering from migraines, the respective migraine health-item is activated. Health-items activated by direct activators are also referred to herein as “explicit health-items” or “reported health-items”. Other types of explicit activators include medical measurements and lab results (e.g., blood test, blood pressure, MRI, CT, etc.) and subjective patient input. For example, lab results which indicate a deficiency in red blood cells is considered an explicit activator which activates a corresponding explicit health-item (e.g., Anemia). Similarly, if a patient reports that he is suffering from bleeding (e.g., when responding to a questionnaire), the data received from the patient is considered an explicit activator which activates a corresponding explicit health-item (e.g., bleeding health-item).

As mentioned above, triggers include possible causes of certain medical conditions. Thus, triggers are a second type of activators (also referred to herein as “derived activators”) which are not directly related to a reported medical condition in the patient's personal health data, rather their effect on medical conditions is deduced by the system. Triggers are used for activating health-items (herein below “derived health-items”) which represent medical conditions which may possibly result from medication errors in the patient's medication regimen and accordingly derived health-items mark potential medication errors and risks. As further explained below, triggers may also be used for supporting the validity of explicit health-items. As part of determining derived activators, information on treatments (e.g., drugs) which are being administered to the patient are retrieved from the patient's personal medical data and information on the ADRs and warnings relevant to these treatments (e.g., drugs) are extracted from the appropriate medical resources. Each ADR or warning represents a potential medical condition that a patient may be currently suffering from, or one that the patient may suffer from in the future.

To identify triggers and respective derived health-items, computer logic is applied (e.g., by health-items activation module in processing circuitry 110) on the medical data which is related to different health-items in the patients' risks-map.

Several examples of computer logic operations carried out during the analysis of the health-items are listed herein below:
Assuming that according to a patient's EMR, the patient is administered with a certain drug X for treating medical condition A, the respective health-item representing medical condition A is activated (explicit health-item.) Assuming further that the medical database 105 links drug X with one or more health-items representing respective medical conditions that may be induced as an adverse effect of the usage of drug X, these health-items (derived health-items) are activated as they represent health risks the patient is potentially facing, and the drug is identified as a potential trigger (cause) of these health-items.
Assuming that according to a patient's EMR, the patient is administered with two different drugs, drug A and drug B, one for treating medical condition A and the other for treating medical condition B, the respective health-items representing medical condition A and medical condition B are activated (explicit health-items). Assuming further that the medical database 105 links these two drugs with one or more health-items representing respective medical conditions that may be induced as a result of a drug-to-drug interaction between the drugs, when taken together, these health-items are activated (derived health-items) as they represent health risks that the patient is potentially facing, and a combination of drugs causing the DDI is identified as a potential trigger of these health-items.
Assuming that according to a patient's EMR, a certain measurement of a blood component in a patients' blood measurement (blood test) is out-of-range and that blood measurement is defined as a possible activator for a certain health-item (representing a respective medical condition), that health-item is activated, its related implications regarding specific drugs administered to the patient are identified, and the out-of-range measurement of the blood component are identified as a potential trigger of the health-item.

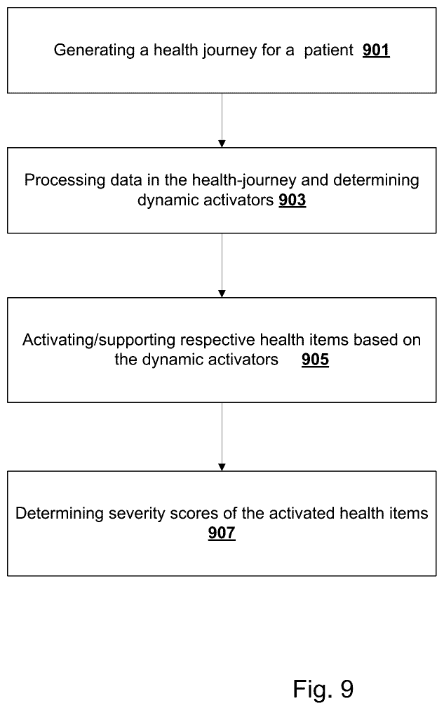

Another type of activators which may be used according to some examples for activating health-items in the risks-map (and calculating their respective validity score as further described below) are “dynamic activators”. These activators are determined based on the progression of data related to health measurements recorded over time (“health journey”). A more detailed description of dynamic activators is provided below with reference to FIG. 9.

The corresponding health-items are activated according to the identified activators and triggers, for example activation may include marking the activated health-items as relevant health-items and adding the activated health-items to the personalized risks-health map. The risks-map can be implemented for example, as a dedicated data structure and/or database, where each entry represents a health-item and stores information on the respective medical condition, respective activators and the personal medical data which is related to the activators (block 409).

Notably, the same health-item may be activated by more than one medical condition. This may occur, for example, where the resolution of common clinical definitions is greater than the resolution of the definitions in the medical database 105 and accordingly a single health-item may represent several related medical conditions. For example, a health-item representing hypotension may be activated by different classes of hypotension (orthostatic, postprandial neutrally mediated, severe, etc.).

Also, multiple medication related indications (e.g., ADRs or warnings) may activate the same health-item. For example, the health-item representing ‘Hypotension’ may be activated by a warning triggered by a medication containing an active pharmaceutical ingredient (API) Losartan (which may be administered to the patient for treating some other medical condition) and also may be activated by a different warning triggered by a medication containing an API Ramipril.

As is well known in the art and was demonstrated above with the example of Amitriptyline, a single drug may be related to many indications including various ADRs or warnings, while only a small subset are relevant to any specific patient. The long list of ADRs and warnings which is commonly disclosed in medication package inserts (also known as “patient information leaflets” or “drug label”) attached to medicines, is often ignored due to their generalization and the difficulty in identifying the ADRs and warnings which are indeed relevant to a specific patient. The system and methods disclosed herein aim to identify relevant ADRs and warnings as part of the generation of the personalized risks-map. The personal medical data of a specific patient is processed to identify potential health risks (including ADRs and warnings) specified in the personal medical data, which may serve as triggers for activating derived health-items and once such data is identified, corresponding derived health-items may be activated.

As the specifics of various risks, including ADR and warnings related to medications and other treatments, depend on many factors such as age, weight, gender, other drugs being used, complexity, renal impairment, medical history, and so on, such factors are taken into consideration during the analysis. As mentioned above, in some examples, the medical database, may include specific data with respect to each of the health-items and triggers indicating the conditions for their activation (referred to herein as “conditions of activation”). The ADRs and warnings are screened according to the patient's personal data, and those ADRs and warnings which are not relevant to the specific patient (e.g., do not comply with the conditions of activation) are removed/ignored. For example, drug related risks which are relevant only to geriatric patients and not to younger individuals, are determined as irrelevant to patients of a younger age group. In another example, a warning related to a certain drug may activate a respective health-item (rendering it relevant) only if another additional indication is reported in the patient's personalized risks-map. For instance, a warning of constipation, associated with a drug reported in the patient's EMR, may activate the “constipation health-item”, only if the EMR also indicates at least one of two specific symptoms. In case none of these two symptoms are reported, the “constipation” health-item is not activated.

In some examples, a derived health-item activated by a trigger, is considered relevant only if there is at least one more activator that supports the trigger. For example, if there are at least two different derived activators that activate the same health-item or if the same health-item is activated by both a derived activator and an explicit activator it is considered relevant.

In some examples, overlaps between explicit health-items and derived health-items are identified and removed to maintain a single instance of each health-item in the personalized risks-health map, thus in some cases an explicit health-item and a derived health-item are merged into a single health-item corresponding to one medical condition.

Thus, the preliminary personalized risks-map includes health-items representing medical conditions which have some type of explicit record in the patient's personal medical data and the patient and/or the medical staff treating the patient may be aware of these conditions, as well as derived health-items representing warnings about potential medical conditions which are not recorded in the patients' medical records and the patient and/or the medical staff may not be aware of their existence. Derived health-items may also help to disclose asymptomatic medical conditions that a patient may have, while being unaware of their existence. Furthermore, derived health-items may also represent health risks which have not yet materialized, thus providing a predictive tool for identifying these risks and recommending interventions to reduce the likelihood of their materialization.

According to some examples, the process of generating a personalized risks-map also includes the calculation of a respective validity score (e.g., by risk assessment module 115) for each relevant health-item (block 411). The validity score (otherwise referred to herein as “validity risk-score”) is a value that indicates a level of certainty that a health-item identified as relevant to the medical status of a certain patient is in fact relevant.

In some examples, only health-items that are assigned with a validity score that is greater than some threshold value, are considered relevant and maintained in the risks-map. In some cases, health-items with a validity score below threshold are removed from the risks-map, where as part of the assignment of validity scores, a screening process is applied where health-items' validity score below threshold are omitted from the map. In other examples, activated health-items are assigned as candidates relevant health-items, and are rendered relevant and added to the risks-map only after it has been confirmed that their validity score is above threshold.

As further explained below, validity scores are one of the core risk-scores which are used for calculating respective health-items risk-scores and a patient risk-score. In some examples validity scores are calculated based on the combination of explicit and derived activators which activate each health-item.

As mentioned above, according to some examples, in addition to the personal patient's medical data mentioned above, subjective patient data is also used by the processing circuitry 110 during the generation of a personalized risks-map for activating and validating health-items. While subjective data may have a critical effect on the evaluation of medication errors and possible risks in the patient medication regimen, it is not readily available and should be retrieved by directly interacting with the patient. Thus, according to some examples, in addition to the personal medical data mentioned above, subjective data obtained from the patient is also used by processing circuitry 110 for generation of a personalized risks-map of a certain patient. The subjective data serves as another type of explicit activators used for supporting the validity of health-items in the personalized risks-map. Subjective data is obtained by direct interaction with the patient for example, by presenting a questionnaire to the patient. Subjective data may be used for further supporting health-items having validity scores below threshold, where, in case the subjective data increases the validity score of a health-item above the threshold, the health-item is retained in the risks-map, otherwise it is removed from the risks-map.

MSIS processing circuitry 110 (e.g., by risks-map generation module 113) can be configured to apply computer logic dedicated to calculating a collective validity score for each relevant health-items, based on the scores assigned to each individual activator of the health-item. In some examples, the validity score depends on the number of activators indicating the relevance of each health-item, where the greater the number of activators, the higher the validity score. One example is the validity score of a derived health-item which is activated by an ADR specified with respect to one drug that is administered to the patient and is further activated (or supported) by blood measurements, found in the patient's EMR, that are known to be common to patients suffering from the medical condition represented by the derived health-item, which may be greater than the validity score of a derived health-item activated by the ADR alone. A second example is the validity score of an explicit health-item activated by a medical condition reported in the patient EMR and is further activated (or supported) by blood measurements, found in the patient EMR, that are known to be common in patients suffering from the medical condition represented by the derived health-item, which may be greater than the validity score of a derived health-item activated by the reported health-item alone. A third example is the validity score of a derived health-item which is activated by an ADR specified with respect to one drug that is administered to the patient and by a drug-to-drug interaction specified with respect two other drugs, which may be greater than the validity score of a derived health-item activated by only the ADR or only the DDI.

The validity score may also depend on the type of activators. For example, activators from different categories (e.g., blood measurement, reported condition, health index) may have different contribution to validation (validation weight) as well. For each medical condition, the respective activators can be each given a respective validity score according to their validation weight, based for example, on the known relation between the activator and the respective medical condition. Thus, the system may include a validity scale, which indicates the validation weight of different activators for validating different medical conditions. A collective validity score of a certain health-item, is calculated based on the combination of the validity scores assigned to each activator. In addition, the initial validity score of a health-item, activated by an explicit activator, is in some examples greater than the validity score of a derived health-item, activated by a trigger (derived activator) as the validation weight of an explicit activator is, in general, greater than that of a derived activator. The validity score may also depend on the specific value of an activator in addition to its type. For example, the correlation between certain blood measurements and a certain medical condition often depends on the specific value of the blood measurement. Thus, a scale can be determined and used for correlation between specific blood test values or ranges and their respective contribution to validity in different health conditions. The validity score assigned to each health-item in the map can be stored for example in the entry representing the health-item in the risks-map.

According to some examples, a severity score is determined for each of the relevant health-items (block 415). The severity score (otherwise referred to herein as “severity risk-score”) is a value that indicates the level of risk of an occurrence of a medical-care event, such as hospitalization and ER admission, resulting from each health-item based on the available clinical knowledge.

In some examples, the severity scores are calculated based on a collection of parameters. These parameters include for example: the body system(s) related to the health-item (e.g., integumentary System, Skeletal System, Muscular System, Nervous System, Endocrine System, Cardiovascular System, Lymphatic System, Respiratory System, Digestive System, Urinary System), the organ(s) related to the health-item (e.g., heart, lungs, limbs, liver, colon, etc.), the specific type of symptoms and the respective measured values and/or severity indexes (e.g., hypertension values, abnormal blood test values, insomnia severity index, etc.). Body systems and organs are known to correlate with medical-care events, for example, in general a cardiovascular event presents a greater risk for occurrence of a medical-care event than itching. In some examples, severity is determined according to various indexes categorizing medical conditions according to their risk to the patient. For example, severity of a high blood pressure condition may vary according to the specific blood pressure measurement values, as higher blood pressure indicates a greater risk of occurrence of a medical-care event.

According to some examples, a severity score is calculated based on a compilation of these parameters. Different parameters can be given a respective severity weight based on a corresponding estimated risk related to the parameter, as known from the medical literature, and a combined severity score can be determined (e.g., as a weighted average) based on the compilation of these weights.

According to some examples, several severity classes are defined, each class representing a different level of estimated impact on the patients' health, possibly resulting in complications and/or hospitalization. For instance, three main severity classes can be defined, where the first severity class represents low impact, the second severity class represents moderate impact, and the third severity class represents high impact. Further subclasses of severity scores can be calculated as well providing intermediate levels of severity.

In some examples, at block 417 medication error/risk assessment of the relevant health-items is carried out. During this stage, the relevant health-items in the patient's personalized risks-map are analyzed to determine medication errors/risks. To this end, computer logic is applied (e.g., by risk assessment module 115) on the medical data which is related to different health-items in the map. As part of the medical risk assessment, health-items are classified as controlled or uncontrolled, where controlled health-items include health-items which are treated and balanced, and uncontrolled health-items include untreated and/or unbalanced health-items. Unbalanced health-items are those which are treated, but the treatment is erroneous (e.g., in dosage or frequency), or ineffective, or lacking.

Examples of computer logic applied during the error/risk assessment of the health-items include:

i. Activated health-items in the personal risks-map of the patient, are matched to information on administered drugs and other treatments obtained from the personal medical data of a patient, to determine whether all medical conditions are being treated. In case it is determined that a medical condition is properly treated with an appropriate drug or some other treatment, the respective health-item is classified as “treated”. Otherwise, the respective health-item is classified as “untreated”, where health-items which are classified as untreated indicate a possible medication error/risk. As mentioned above, information regarding the drugs which are administered to a patient for treating specific medical conditions can be retrieved for example from the patient's personal medical data.

In some examples, a matrix can be generated for comparing all health-items in the personalized risks-map of a patient to find a matching drug or other treatment for each medical condition represented by the health-items. If a certain drug is known to be used for treatment of only one medical condition, and the patient has that condition, a match can be determined, and the respective health-item is classified as treated. If a certain drug is used for treating two different medical conditions, and the patient has both, additional information obtained from the personal medical data of the patient can be used for determining which of the medical conditions is being treated using the drug. For example, if the EMR indicates a second drug that is used for treating one of the two medical conditions, it can be determined that the first drug is used for treating the remaining medical condition.

ii. Information on administered drugs and other treatments obtained from the personal medical data of a patient are further analyzed to determine whether the respective drug administration regimen is in accordance with the common medical recommendations provided by physicians. This includes, for example, dosage, frequency, delivery (e.g., oral, or topical), etc. The specifics of the treatment are compared to the recommendations, as presented in the medical database 105. In case a health-item is found to be balanced, i.e., the treatment (e.g., drug administration) is according to the specifications defined for the patient, no derived intervention (or “insight”) is triggered. Otherwise, in case if a discrepancy is found between the treatment and the recommendations, the health-item is found to be unbalanced, an intervention (insight) is triggered.

iii. A patient's lab results which are defined as out of range for a specific health-item, may indicate that the health-item is uncontrolled. For example, lab results showing blood pressure above threshold in the last 3 months, may indicate that a “hypertension” health-item is uncontrolled.

In some examples, controlled health-items are assigned to a low-risk class indicating little or no impact on possible deterioration in the patient's condition which may lead to occurrence of a medical-care event and uncontrolled (including unbalanced or untreated) as well as derived health-items are assigned to either a moderate impact class or high impact class, depending on their estimated impact on the occurrence of a medical-care event.

The results of the error/risk assessment of each health-item in the map, including the classification of each health-item and the reason for the classification, can be stored in the entry representing the health-item in the risks-map data structure.

An example of a balanced health-item is an activated high blood pressure health-item, which includes: 1) a data element showing that the patient is being administered with a medicine for treating high blood pressure, where the drug administration details are according to the common recommendations; and 2) data elements showing information on recent blood pressure measurements values that are within the acceptable range.

Notably, as the specifics of various medications and other treatments depend on many factors such as age, weight/BMI, gender, other drugs being used, medical history, and so on, these factors are taken into consideration during the analysis (available for example in the medical database 105). For example, correct dosing of administered drugs is evaluated according to age and weight/BMI based indexes and/or existence of renal impairment. In another example, some drug related risks are relevant only to geriatric patients and not to younger individuals. The complexity of the patients can also be considered, where, in case the patient is a complex patient and/or suffers from renal impairment, the measurements and values are adapted to match the specific values recommended to such patients.

Furthermore, in some examples, health-items in the personalized risks-map are divided into subsets, each subset representing a category of health-items that share one or more common attributes (e.g., clinical/health related, body-system related, organ related) (block 419). In some examples, health-items assigned to categories can be further assigned to sub-categories (e.g., according to the specific organs). A non-exhaustive list of examples of categories include:

Respiratory system, Digestive system, Skin, Movement, Mental and behavioral disorder, sleep disorder, and so on. By way of example, sleep disorder may include all health-items related to sleep such as, insomnia, narcolepsy, and the mental and behavioral disorder category may include heath items such as: depression, anxiety, sleep deprivation, lack of appetite, etc.

Reverting to block 303 in FIG. 3, the personalized risks-map that has been generated includes one or more relevant health-items, each assigned with a respective validity score and severity score. In some examples, once the (preliminary) personalized risks-map is available, interventions are generated (block 305). To this end computer logic can be applied (e.g., by intervention engine 117) for the purpose of analyzing the previously identified medication errors or risks in the personalized-risks-map, and suggesting possible interventions dedicated to remedying these errors.

One should recall that health-items in the risks-map can be divided into a number of types, including explicit health-items which are controlled, explicit health-items which are uncontrolled (including “untreated” and “unbalanced” health-items) and derived health-items. Interventions are applied on health-items which are identified as related to some health risk, generally including uncontrolled explicit health-items and on derived health-items (referred to collectively as “risk related health-items”).

In case all health-items in a personalized risks-map of a certain patient are classified as controlled explicit health-items and it is determined that no health risks have been identified, then in such cases no intervention may be required. In some examples, a message can be generated and provided e.g., to the clinician device, indicating that no interventions are needed. In some cases, even if all health-items are controlled, preventive interventions may be triggered based on the patients' attributes (e.g., age, gender, comorbidities). In other cases, if all health-items are controlled but some of the medications are not within the recommended dosage ranges, an intervention to change the medication dose may be triggered.

Interventions are generated for risk related health-items in an “intervention subset” of health-items. In some examples, the intervention subset includes all risk related health-items found in the personalized risks-map. In other examples, the intervention subset includes only part of the risk related health-items. For example, only to risk related health-items which are assigned with a validity score and/or severity score which is above a certain predefined threshold value and/or only to ‘n’ risk related health-items, ‘n’ being some integer, with the highest validity scores and/or highest severity scores. For this purpose, in some examples some function can be applied (e.g., an average or a weighted average) on the validity score and severity score of each risk relevant health-item to obtain a combined value that can be used for selecting the risk related health-items for which interventions are generated.

At block 305 interventions are generated. To this end computer logic can be applied for identifying errors or risks associated with risk related health-items (e.g., assigned to the “intervention subset”) and for suggesting possible interventions dedicated to remedying these errors and alleviating the related risks (e.g., by intervention engine 117). Interventions are generated with respect to the medications or treatment that are found to be the source of a health risk or error (e.g., based on information determined as described above with respect to block 417).

As part of generation of the interventions, intervention engine 117 can be configured to process the information in the health-items in the intervention subset, determine the cause for an identified risk or error and accordingly provide recommendations for changes in the patient's health regimen dedicated to reducing the related risks and/or amending errors.

A non-exhaustive list of examples of possible recommendations for intervention includes:

A first type of intervention is “replace drug”, recommending changing one or more drugs which are being administered to the patient. For example, in case a derived medical condition is triggered by an adverse drug reaction, changing the related drug may be recommended. In case a derived health-item is triggered by a drug-to-drug interaction, changing one of the related drugs may be recommended. In case a certain drug is used for treating a health condition represented by an explicit health-item, and lab results indicate that the drug is ineffective, a replace drug intervention may be triggered.

A second type of intervention is “add drug” or “remove drug”, recommending adding a new drug or removing an existing drug from the currently administered medication regimen. Adding a drug may be recommended for example in case of an uncontrolled condition where a currently prescribed medication is at the maximum dose recommended for the patient based on the guidelines. Removing a drug may be recommended if a patient is consuming a drug with no current need based on his clinical conditions and lab results. As further explained below, each intervention is assigned with a respective intervention score (otherwise referred to as “intervention risk-score”).

A third type of intervention is a recommendation to reduce a dosage of a drug (e.g., due to overtreatment of a condition) or increase the dosage (e.g., due to lack of effectiveness).

A fourth type of intervention may be a recommendation to monitor the patient regarding a specific measurement or symptom that may be associated with an identified risk related to a drug.

In some cases, a recommendation for an intervention may not be conclusive. For example, assuming a health-item is associated with three different drugs, where all three drugs have a similar adverse affect on the patient, it may be difficult to determine which one is the cause of a certain observed symptom. Thus, in some cases, the recommendation includes a few options, leaving it to the discretion of the medical practitioner to decide which intervention to follow.

An example of the processing applied on the personalized risks-map is provided herein below:

### Health-Item A

One health-item in the risks-map is an explicit health-item A representing medical condition A, which is being treated by administering drugs a1 and a2. As explained above, this health-item is activated based on information disclosed in the patient's personal medical data. The error/risk assessment processing indicates that health-item A is controlled, i.e., drugs a1 and a2 are being properly administered for treating medical condition A.

### Health-Item B

A second health-item in the risks-map is a derived health-item. Health-item B was activated for representing a potential risk of medical condition B, which is an ADR of the use of drug a1 (drug a1 being a trigger of derived health-item B). Health-item B was further validated by blood test results which support the possibility that the patient is suffering from medical condition B and therefore its validation score is above the threshold to be considered validated (enter the intervention subset).

### Health-Item C

A third health-item in the risks-map is an explicit health-item C representing medical condition C, which is being treated by administering drug c. The error/risk assessment indicates that health-item C is uncontrolled. In this case, the error/risk assessment processing found that the patient is suffering from renal impairment while the dosages administered to the patient are according to individuals who are not renally impaired.

### Health-Item D

A fourth health-item in the risks-map is a derived health-item D. Health-item D was activated for representing a potential risk of medical condition D, which may result from the interaction between drug c and drug a2 (the drug-to-drug interaction between the drugs being the trigger of derived health-item D). Health-item D was not validated by another activator and is therefore classified as an open health-item that requires further validation. Subjective data is obtained from the patient during application of the questionnaire indicating that the patient is not suffering from medical condition D, and is therefore classified as not relevant.

### Health-Item E

A fifth health-item in the risks-map is an explicit health-item E representing medical condition E. The patient's EMR does not indicate any medication is being prescribed for treating the medical condition E (or in another example, the proper medication is prescribed, but with incorrect dosage). The error/risk assessment processing indicates that health-item E is uncontrolled as a supporting lab result related to health-item E is identified.

According to this example, recommendations for intervention may include:
A recommendation to replace drug a1 with an alternative drug which is not characterized by the same ADR. Notably, the drug has been replaced, although the health-item is classified as controlled.
A recommendation to adapt the dosage of drug c in compliance with drug dosing of the renal impairment index.
A recommendation to start treatment of health condition E with an appropriate medication.

Referring to blocks 315, to 327 in FIG. 3, risk-scores are generated for the patient based on the data gathered in the patient's personalized risks-map. The calculated risk-scores can serve in practice as a risk prediction scoring system, where the score(s) indicate the magnitude of the risk to the patient of experiencing a medical-care event.

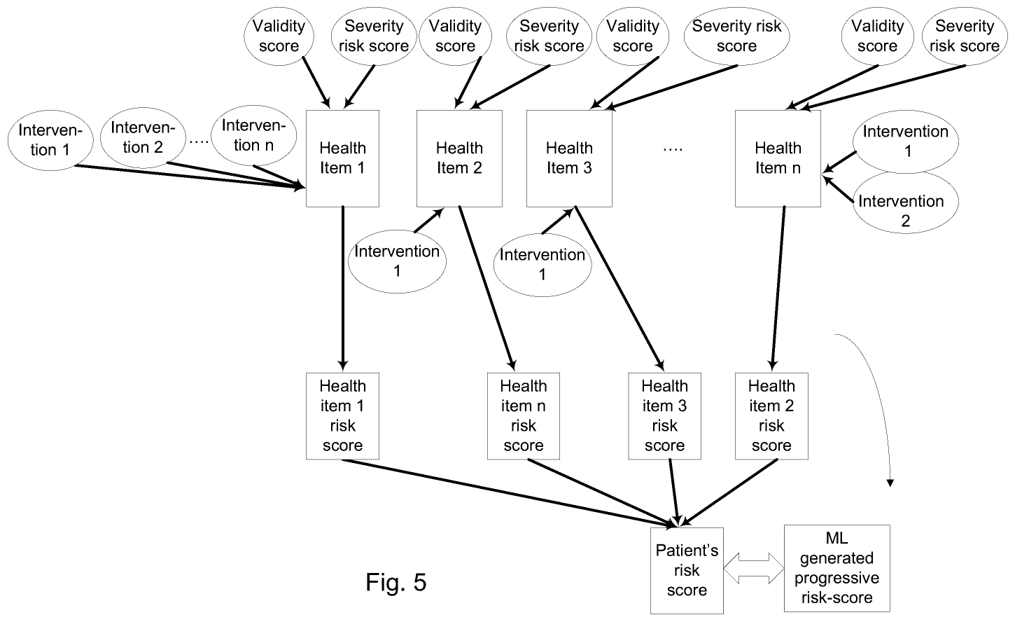

FIG. 5 shows a schematic illustration of data generated as part of the personalized risks-map. FIG. 5 illustrates health-items 1 to n being part of a personalized risks-map generated for a certain patient. As explained above and schematically illustrated in FIG. 5, several parameters are defined with respect to each health-item, including:
Severity score—defining an estimated severity for a health risk that is related to the respective health condition. The severity score indicates the severity of a medical-care event resulting from the respective health condition.
Validity score—indicative of the level of certainty that the patient suffers from the related health condition. The validity score is based, for example, on the type of activators and/or the number of activations and/or the specific combination of the activators.
Intervention score—a respective score is determined for each of the generated interventions (block 315). As explained above, there are different types of interventions, which can be applied for modifying the medication regimen. According to some examples, each intervention type has a respective score indicating its impact on the patient's health assuming the intervention is not complied, and specifically on the risk of occurrence of a medical-care event that is related to the intervention. For example, the risk of occurrence of a medical-care event in case the patient ignores a “replace drug” intervention may be very high. Accordingly, each intervention type is assigned with a respective “intervention-type risk-score”. For example, remove drug, may be assigned with a first score, add new drug with a second score, replace drug with a third score, reduce dosage of existing drug with a fourth score, increase dosage of existing drug with a fifth score, and so forth.

Notably, interventions can be generated with respect to certain drugs or treatments and accordingly more than one intervention can be generated for the same health-item. For example, if a certain health condition is treated by two drugs, an intervention can be generated for each one of the drugs, giving rise to two interventions for the same health-item. By way of example, FIG. 5 shows health-item 1 having 3 respective interventions, health-item n having 2 respective interventions and the other health-items having each, one respective intervention. In some examples, if no intervention is provided to a health-item, the intervention risk-score is set to 0.

Given the different scores calculated for each relevant health-item (including severity score, validity score intervention score, collectively referred to herein below as “core risk-scores” or “basic risk-scores”) according to some examples, they are used for calculating a respective health-item risk-score for the respective health-item (block 317). As each of the core risk-scores is indicative of a specific risk-related attribute characterizing the personal risks-map (validity, severity, and interventions), the health-item risk-score calculated from the core risk-scores provides a compiled risk value that is based on the combination of these attributes. Furthermore, where each of the core risk-scores is determined in the context of the risk of medical-event occurrence, the health-item risk-score provides a relative value which indicates a compiled risk of occurrence of a medical-care event resulting from the respective health condition represented by the health-item.

A health-item risk-score can be calculated using some mathematical function that takes as input the different core risk-scores assigned to the health-item. According to one example the function can be based on the multiplication of the plurality of core risk-scores assigned to the health-item. According to another example the function can be a geometric mean of the core risk-scores assigned to the health-item. According to another example the function can be an average of core risk-scores assigned to the health-item. The functions can include in some examples, weights which are assigned to each one of the different core risk-scores. In further examples, other (e.g., more complicated) functions can be used instead. Notably, if more than one intervention is provided for the same health-item, the respective health-item risk-score can be calculated from the aggregation of the risk-score of all interventions (e.g., accumulation of the interventions risk-score values).

The health-item risk-scores classify the health-items in a personalized risks-map according to their respective related risk and can be used for example to sort the health-item and identify the respective health conditions which pose the greatest risk to the patient. This enables a medical practitioner to identify the most urgent health problems and treat them first. For example, health-items risk-scores can be on a scale of 1 to 1000, providing a normalized scheme for comparing between the relative risks of the health-items in the risks-map.

In some examples, once a respective risk-score is calculated for the health-items in the risks-map, a collective risk-score is calculated from all the calculated health-items risk-scores (referred to herein as “patient risk-score”; block 319). This risk-score provides a value representing an estimated risk of the patient derived from the patient's personalized risks-map. The patient risk-score can be used as a comparative tool for comparing between different patients and identifying patients which exhibit different risk levels.

In some examples, the output of the risk stratification and intervention process includes the (final) personalized risks-map, which includes the relevant health-items, the risk-score values assigned to the health-items, and the interventions recommended with respect to drugs and/or treatments in the patient's medication regimen (block 325). This output can be provided to a medical practitioner such as a doctor or pharmacist and/or to the patient (block 327). For example, recommendations for intervention and/or patient risk-scores can be presented to a medical practitioner via a dedicated interactive tool, or can be embedded in general purpose Electronic Medical Records based tools. Thus, the presently disclosed subject matter provides a computer implemented automatic tool (method and system) for identifying and prioritizing treatment for a specific patient.

Interventions can be classified as high priority/low priority or rated according to their respective risk-score. A medication risks-map can be generated based on these classifications highlighting (e.g., by color coding) the associated risk of each drug in the patient's medication regimen according to the respective risk-scores of the interventions.

The information gathered in the (final) personalized risks-map can be provided to a medical practitioner, e.g., in the form of table listing all the relevant health-items and specifying, for each health-item in the table related information including for example part or all of the following data: a corresponding health-condition, whether it is a derived or an explicit health-item, whether it has a risk-related to it or not, the related interventions and the reasons for each intervention, a respective health-item risk-score, a respective validity score, a respective severity score, the intervention risk-scores, prioritization of the interventions, etc. Thus, providing a clear and convenient tool for informing the medical practitioner about the medical status of a patient, and particularly about identified medical risks and interventions recommended for alleviating these risks.

The patient risk-score can be used for classifying different patients according to their respective risk, providing different warnings of different severity according to their risk and/or prioritizing the execution of interventions according to the patient risk-score. For example, the risk stratification and intervention process can be applied on a population of patients (e.g., the population of patients in a retirement home, polypharmacy patients in a community setting, all chronic patients above 65 in a community setting, or all patients in a certain city or district, etc.) and provide a respective risks-map and patient risk-score for each patient in the cohort. The patient risk-score can be indicative of the urgency of intervention, where a relative high patient risk-score indicates to a medical practitioner or other staff member that urgent intervention is required. Thus, the presently disclosed subject matter provides a computer implemented automatic tool (method and system) for identifying and prioritizing treatment for patients within large cohorts. More specifically, it allows to classify/prioritize patients according to their risk of experiencing a medical-care event and provides intervention to high priority patients that exhibit high risk to avoid or reduce the likelihood of the occurrence of such an event.

For example, patients' health-risks stratification module 123, can be configured to manage the process where personal medical data of multiple patients of a certain population (cohort) is processed to determine a respective patient's risk score for each patient and provide the classification of the patients according to their assigned risk-score and other risk related attributes (e.g., affinity groups based on location, age groups, health quality metrics, etc.).

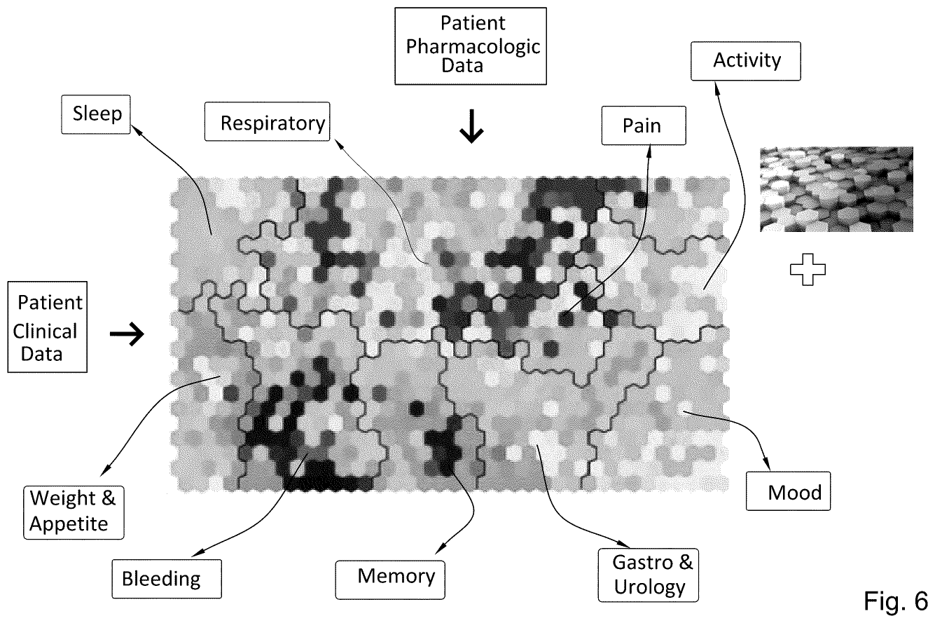

In some examples, the personalized risks-map is graphically displayed. FIG. 6 is a schematic illustration showing a graphical representation of a personalized risks-map, according to an example of the presently disclosed subject matter. As shown in the illustrated example, the relevant health-items are graphically represented as hexagons, where the collection of hexagons provides a graphical representation of the personalized risks-map. Graphical representation can be provided instead or in addition to textual representation.

The graphical representation of the map can further include information graphically displaying the classification (e.g., controlled, uncontrolled, treated, untreated), severity, and validity of the health-items. According to one example, the severity and validity score of a certain health-item in the map can be displayed on the screen, e.g., responsive to a mouse hover over the health-item. Furthermore, in some examples, the graphical display may include indication of the intervention scores and/or the health-items risk-scores. For example, color-coding scheme of the different risk classes can be provided where each class corresponds to a certain range of risk-score values. In one example, green color is used to mark health-items classified to a low-risk-score class, i.e. health-items that represent medical conditions which have little or no potential of impacting the patients' health, such as balanced health-items, red color is used to mark health-items classified to a high risk-score class; and yellow color is used to mark health-items classified to moderate risk-score class. Additional colors such as blue, light blue and yellow can be used to mark various intermediate classes residing in between the three main classes mentioned above. FIG. 6 also shows the division of the relevant health-items into different health categories. The graphical representation of the map can be displayed on a display device to be viewed by a user e.g., on end device 130 and/or 131.

Following the description of the generation of a personalized risks-map, the generation and usage of patient health-journey is now disclosed. The term “health-journey” (“HJ” in short) is used to describe the history of the personal medical data of the patient over time. Examples of personal medical data are listed above, with respect to FIG. 4. Personal medical data further includes information about “medical-care events” and “patient's behavioral data”. The phrase “medical-care event” refers to acute health-care evets that occurred to a patient, such as hospitalization and ER admissions. Patient's behavioral data describe the behavior of the patient in the context of his health, for example, whether a patient purchased a prescribed medication or not, whether the patient took (“adheres to”) the prescribed medication or not, whether the patient went to a specialist, etc. Patients' behavioral data may further include various types of ambulatory health-care events including for example, recorded doctor visits, imaging procedures, such as MRI, XRay, CT, (in some examples, here their occurrence and frequency is addressed, not the results), etc. Information on “Medical-care events” and “patient's behavioral data” can be obtained for example, from the patients EMR.

The health-journey is comprised of historical and current personal medical data collected over a certain health-journey period, e.g., personal medical data going back to the past 2, 4, 6, 12, 18 or 24 months. Different types of personal medical data are processed and correlated to obtain one synchronized picture of the progression and changes in the personal medical data over the health-journey period.

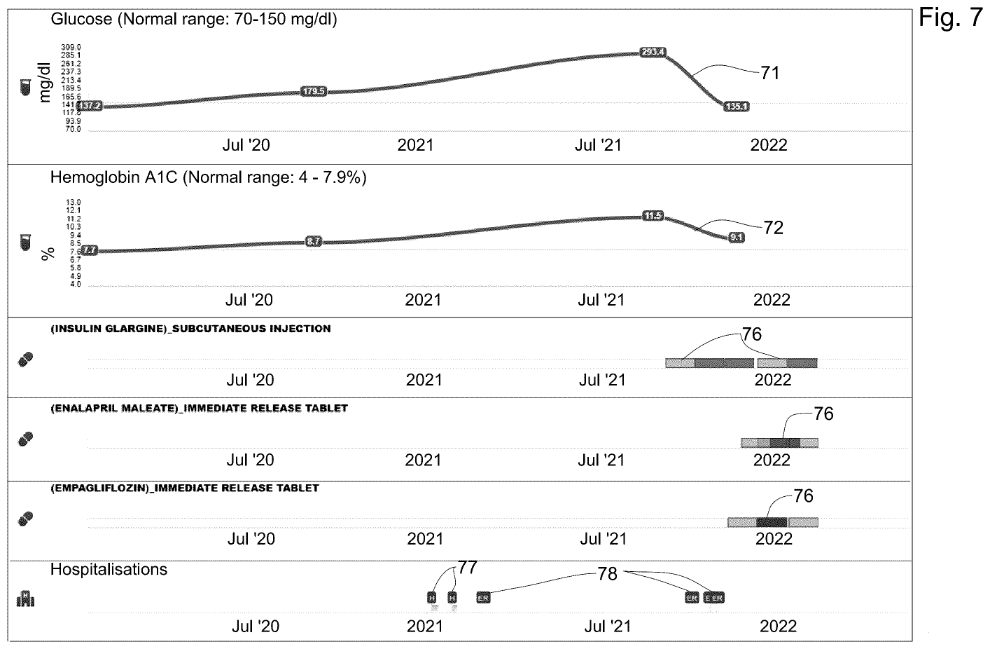

FIG. 7 shows an example of graphical representation of some components of a health-journey, according to some examples of the presently disclosed subject matter. FIG. 7 shows a first progression graph (71) of a patient's glucose levels and a second progression graph (72) of the patients' Hemoglobin A1C levels, recorded over a timeline (the health-journey period) extending between February 2020 and December 2021.

FIG. 7 also shows three drugs prescribed to the patient, Insulin Glargine (73), Enalapril Maleate (74), and Empagliflozin (73), along with additional information with respect to drug related events, such as initial prescription date (76) along the health-journey period, and adherence indicated by color coding, where light color (e.g., green) indicates that the drug has been prescribed and purchased and dark color (e.g., red) indicates that the drug has been prescribed but not purchased. FIG. 7 further shows information on medical-care events, including hospitalization events (77) and ER admissions (78) and their occurrence along the health-journey period.

A health-journey is used for monitoring changes in the health condition and medications/treatments of patients over longer periods and for deducing more accurate and robust prediction of deterioration and hospitalization risk than the prediction made based on a single risks-map. As further explained below, according to some examples of the presently disclosed subject matter, health-journeys are used for generating a “risks-maps-sequence”. A risks-maps-sequence is generated from a plurality of personalized risks-maps, where each risks-map instance is generated with respect to a certain time point along the health-journey period, based on personal medical data which is available with respect to the certain time point. By repeating the generation of the personalized risks-map at different times a series of instances of personalized risks-maps (at least two) is obtained, each risks-map representing the specific medical picture of the patient at the specific time it was generated. By way of example, assuming the health-journey period is 6 months long, a personalized risks-map can be generated for each month along the period, giving rise to 6 risks-maps or for every two weeks giving risk to 12 risks-maps, etc. A detailed description of the process of generating a personal risks-map is described above (see for example, FIGS. 3 and 4).

According to some examples, each personalized risks-map is represented as a node in a data-structure (e.g., graph, linked-list, or the like), providing a grid of personalized risks-maps over time, each node being associated with an object or objects comprising information regarding the relevant health-items in the respective risks-map. As further explained below, risks-maps-sequences are generated and processed for further enhancing the risk-stratification and intervention process. As part of the analysis of the health-journey the risk-scores of the health-items are calculated not only based on information extracted from a recently updated risks-map (current risks-map) but also based on information extracted from a succession of risks-maps constituting the risks-maps-sequence. As further demonstrated below risks-map-sequences represent a novel entity (e.g., data-structure) which is specifically designed as input for training and execution of the Machine Learning model, where once trained, the Machine Learning model can accurately classify patients according to their respective risk of experiencing a medical-care event.

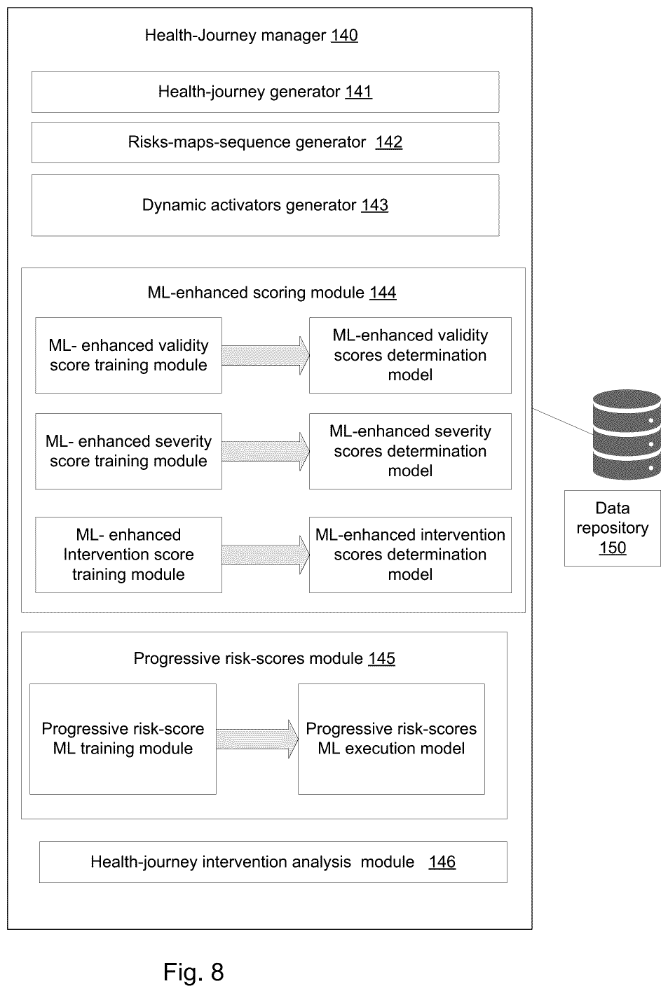

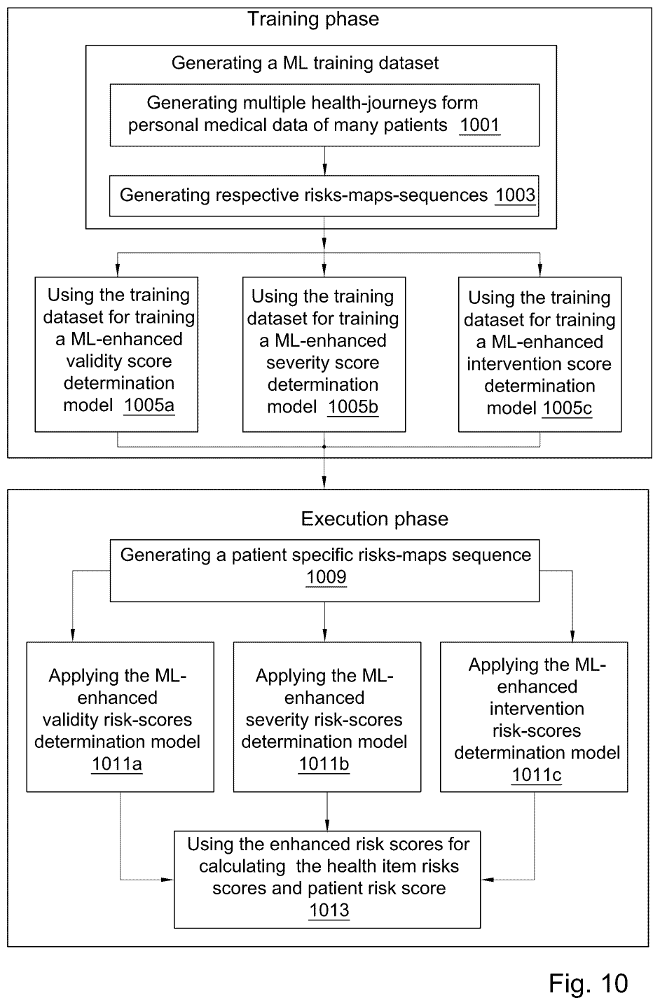

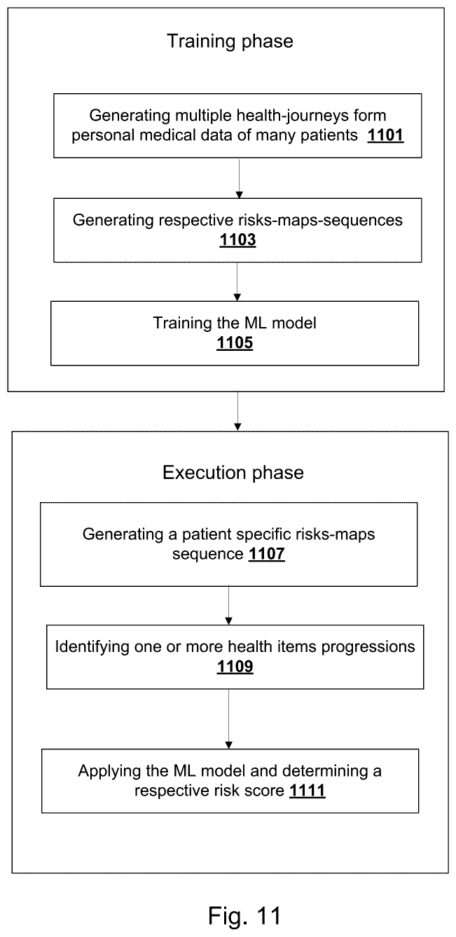

Reverting to FIG. 2, in some examples it can further comprise or be otherwise operatively connected to a health-journey engine 140 configured to generate and process health-journeys as further disclosed below with respect to FIGS. 9 to 12. FIG. 8 is a block diagram schematically illustrating a more detailed view of some components of the health-journey engine 140, according to some examples of the presently disclosed subject matter. Notably, health-journey engine 140 can be implemented on a device separated from, but operatively connected to, server 107 and/or processing circuitry 110. Health-journey engine 140 (implemented for example as a dedicated processing circuitry comprising one or more computer processors) comprises health-journey generator 141 configured to generate health-journeys and risks-maps-sequence generator 142 configured to generate a risks-maps-sequences from the respective health-journeys. To this end, health-journey generator 141 can be operatively connected to patients' data resources 103 or to some other data resource (e.g., designated database 150) storing historical and updated personal medical data of patients. According to some examples, risks-maps-sequence generator 142 can be operatively connected to personalized map generation module 113 and use it for generating multiple personalized risks-maps for different patients. Health-journey manager 140 can further comprise dynamic activators generator 143 configured to apply computer logic on the generated health-journeys and determine dynamic activators as further described below with respect to FIG. 9. Health-journey manager 140 can further include ML-enhanced scoring module 144 configured to apply Machine Learning (ML) model(s) on the generated risks-map sequences for determining enhanced risk-scores values as further described below with respect to FIG. 10. Health-journey manager 140 can yet further comprise progressive risk-score module 145 configured to apply Machine Learning model(s) on the generated risks-maps sequences for identifying ML-enhanced patient risk-scores (also referred to herein as “progressive risk-scores”), as further described below with respect to FIG. 11. Health-journey manager 140 can be operatively connected to a database (e.g., database 129/150) configured to store the processing output products of the various modules.

The following description provided with respect to FIGS. 9, 10, 11, and 12 shows various examples of methods that enable to enhance the accuracy of the risk stratification and intervention process based on health-journey analysis. As mentioned above, these methods can be applied on a large population of patients for the purpose of classifying patients in the population according to their assigned patient's risk-score and other specific health related attributes. The disclosed method and system therefore enable automatic identification of patients, in large populations, that are at high medical risk, specifically risk of experiencing hospitalization or some other medical-care event. By taking appropriate measures (e.g., applying the suggested interventions) these risks can be reduced, and the medical-care event can be avoided.

Attention is now drawn to FIG. 9 showing an operation flow of health-journey analysis for determining dynamic activators, according to some examples of the presently disclosed subject matter. Operations described with reference to FIG. 9 can be executed by way of example, by health-journey manager 140, and more specifically by dynamic activators generator 143.

As explained above, health-item activators are used during the generation of a personalized risks-map for activating and supporting the validity of activation of certain health-items thus increasing the estimated probability that the respective health condition represented by these health-items are relevant to the patient's health. Activated health-items (e.g., having a validity score above some threshold) are rendered relevant health-item and are added to the patient's personalized risks-map. Different types of medical data (clinical, pharmacological, and subjective) are used as activators. Taking blood pressure values as an example, high blood pressure values can be related to a hypertension health condition and thus serve to activate “hypertension” health-item, but also can serve to support the activation of other health-items related to conditions which involve increased blood pressure. As mentioned above, as part of the risk stratification and intervention process, health-items risk-scores and a patient risk-score are calculated based on basic risk-scores which include the validity score.

Another type of health-items activators (referred to herein as “dynamic activators”) that are determined, based on a patient's health-journey, as part of the risks-map generation process is disclosed herein below. The generation and application of dynamic activators can be done as part of the flow described above with respect to FIGS. 3 and 4, e.g., as part of the operations in block 405 and 417.

As explained above, a health-journey is generated from personal medical data obtained over a certain period (block 901). The health-journey is processed to determine dynamic activators (block 903). A dynamic activator is comprised of a sequence of values of a specific type of medical data, recorded in a health-journey over time, where each value is recorded at a different time along the health-journey period. This includes the observed clinical and pharmacological data attributes such as health conditions, medical measurements, subjective inputs, API/MoA, dosing, frequencies, etc.

Referring to FIG. 7, each blood test result (71, 72) has a distinctive pattern, defined by the progression, trend and magnitude of the values measured at different times along the health-journey period, as shown in the respective graphs, where “progression” refers to the frequency of occurrence of each measurement and “trend” refers to the changes in the values over time.

In the example of health conditions, the health-journey may include an occurrence pattern indicating whether a health condition appears consistently (e.g., headaches are recorded at each sampled time point) or sporadically along the health-journey and for how long. In the example of subjective inputs, the health-journey may include a subjective input pattern, indicating whether a patient's complaint about a certain health problem is consistent or sporadic and for how long. In the example of frequency and dosage, the health-journey may include a pattern showing changes in frequency or dosing of medications prescribed to the patient (which may result from either change in prescription or from patient's adherence).

According to some examples, rule-based computer logic is applied (e.g., by dynamic activators generator 143) on the health-journey for identifying dynamic activators and determining their respective correlation with specific health conditions. The rule-based computer logic can be generated for example, based on medical information which can be obtained, for example, from the available medical literature.

Once determined, dynamic activators are used for activating respective health-items or supporting respective health-items previously activated (block 905). Dynamic activators increase the variety of activators which are available for activating and supporting the activation of health-items and thus increase the robustness and accuracy of the generated risks-map. Furthermore, specific attributes characterizing each sequence of values (or “pattern”) are used to differentiate between dynamic activators of the same type of medical data and provide a more accurate activation scheme, in which different dynamic activators of the same type of medical data, may each activate a different health-item, depending on their respective pattern. The use of dynamic activators enables to determine a more accurate and specific distinction between related health conditions, which can be represented by respective health-items in the risks-map, thus increasing the resolution and accuracy of the risk-stratification and intervention process.

Information on dynamic activators indicating a correlation between dynamic activators (each characterized by a specific type of medical data and a distinctive pattern) and respective health conditions can be stored in data-repository, which is made accessible to dynamic activators generator 143. During operation, once a dynamic activator has been identified, the data-repository can be searched for an identical or similar dynamic activator to thereby identify a respective health condition which is known to exist in correlation with the dynamic activator. If such health-item is identified, it is activated or further supported if it has already been activated. Similarity between the dynamic activator found in the health-journey and one which is found in the data-repository can be based on various features extracted from the dynamic activator as well known in the art.

Considering glucose level as an example, assuming three different patients were tested for glucose levels on the same day, and all tests showed the same glucose level above a threshold, which may indicate diabetes, and therefore activate the respective diabetes health-item. However, when observing the glucose measurement patterns recorded over the same health-journey period, significant differences in the glucose measurements pattern of each patient are observed. A first patient exhibits steady values of glucose (above a threshold) throughout the health-journey period, a second patient exhibits glucose levels which are below a threshold at the beginning of the health-journey period, and an abrupt and continuous increase in the glucose levels starting at some point along the health-journey period, and the third patient exhibits fluctuating glucose level values, rising above and then falling below the threshold value. In the above example, each pattern of glucose level measurements may represent a distinct activator of a respective health-item, where each health-item represents a specific health condition that is known to be associated with a certain pattern of glucose level measurements. The computer logic is applied for analyzing the glucose level measurements (dynamic activators), determining their pattern, and activating the appropriate health-item.

Considering blood pressure as another example, the observed pattern of blood pressure measurements over the health-journey period can be compared to a hypertension threshold, where blood pressure above this threshold is considered to indicate hypertension, and the changes in the blood pressure values relative to the threshold are determined. A respective dynamic activator is determined according to the trend of the blood pressure values along the health-journey. For example, whether the blood pressure values are constant, or show a steady increase over time, or a steady decrease over time. The dynamic activator may also depend on the characteristics of the change in values, for example the rate in increase or decrease in values. In some cases, the blood pressure values may fluctuate above and below the hypertension threshold, while in other cases the blood pressure values may fluctuate, rising above the hypertension threshold and then below a hypotension threshold. In each of these cases the computer logic may determine a different dynamic validity activator, activating one of many health-items, each representing a different health condition related to hypertension that is known to be associated with a specific blood pressure measurements pattern.

In addition to correlating between individual types of medical data observed in the health-journey and specific health conditions, in some examples, the computer logic correlates between specific combinations of different types of medical data. For example, a specific pattern of hemoglobin A1C measurements may activate one type of health-item representing a first health condition, if it is accompanied by an indication of a drug intake that is known to include a certain API and may activate another type of health-item representing a different health condition if no medication intake is found.

In some examples, following determination of dynamic activators, a severity score is assigned to health-items activated by the dynamic activators (block 907). As explained above with respect to block 415, health-items are assigned with a severity score that indicates a medical severity or medical risk (e.g., hospitalization risk) that the specific health condition related to the health-item presents to the patient. As dynamic health-items enable to determine a more accurate and specific distinction between related health conditions, each of these distinct health-items can be assigned with a severity score, based on the known risk related to the respective health condition. For example, reverting to the example of increased sugar levels mentioned above, each sugar levels pattern activates a different health-item which is assigned with a different severity score that is defined according to the risk related with the respective health condition. Thus, by increasing the resolution of health-items, the dynamic activators also enable to provide more accurate severity scores to patients, based on the more accurate determination of their health condition, and accordingly more accurate health-item risk-scores.

As mentioned above, dynamic activators can be used as part of the process of risks-map generation. Once the respective health-items have been activated and their validity score and severity score has been determined, the process can proceed to determining interventions, interventions risk-scores, health-items risk-scores and patient risks scores as explained above with respect to FIG. 3.

FIGS. 10-12 demonstrate the advantages which are obtained from the transformation of the raw data in the health-journey to risks-maps-sequences. Machine Learning models are illustrated, which are not applied on the raw medical data of the patient but rather on the health-items in the risks-maps-sequence. The generation of the risks-maps that models the health conditions of patients is therefore an essential step in these processes, however calculation of the core risks-scores is not always necessary.

Attention now reverted to FIG. 10 showing an operations flow of Machine Learning analysis for determining ML-enhanced core risk-scores, according to some examples of the presently disclosed subject matter. By way of example only, operations described with respect to FIG. 10 can be executed by health-journey engine 140 and more specifically by ML-enhanced scoring module 144. The generation and application of ML-enhanced core risk-scores can be done as part of the flow described above with respect to FIG. 3 (block 321). Notably, although the training of the Machine Learning models and their execution is described in a single flow, this is done for ease of understanding and simplicity only. The training of the Machine Learning model and its execution can be performed asynchronously and/or by different computer devices, possibly located remotely one from the other. It is further noted, that although the generation of different types of ML-enhanced core risk-scores are described in a single flow, each type can be generated in a separate process which trains and uses specific Machine Learning models and, in some cases, only part of ML-enhanced core risk-scores may be used.

As explained above, core risk-scores are calculated for each health-item. This calculation is performed using rule-based computer logic that defines how to assign risk-scores to each health-item based on clinical knowledge. These include validity scores, severity scores and intervention scores. According to further examples, Machine Learning models (implementing for example regression algorithms) are used for processing patients' risks-maps-sequences and determining a new type of scoring scheme including ML-enhanced validity scores, ML-enhanced severity scores and ML-enhanced interventions scores (referred to herein collectively as “ML-enhanced core risk-scores”). As further discussed below, Machine Learning models are trained to determine the ML-enhanced core risk-scores based on correlations identified in risks-maps-sequence and the observed medical outcome and thereby improve the accuracy and resolution of the risks-scores and obtain more accurate health-item risk scores and patient risk-scores, which are derived from them.

To this end initially a ML training dataset is generated. At block 1001 personal medical data of many patients (e.g., from thousands up to millions of patients) is obtained. The personal medical data includes historical medical data collected over a certain period (health-journey period), e.g., personal medical data from the past 4, 6, 12, 18 or 24 months, which constitutes the patients' health-journeys. Patients' health-journeys also include medical outcome data indicating occurrence of medical-care events such as hospitalization and ER admission along the health-journey period, and possibly also data indicating the medical condition(s) that was the reason for the hospitalization or ER admission. At block 1003, corresponding risks-maps-sequences are generated from health-journeys, which constitute the training dataset and includes the data on the medical outcome. Each risks-map is generated with respect to a certain time point along the health-journey period, based on personal medical data which is available with respect to the respective time point. Changes in the patient's personal data along the health-journey are reflected as changes in different risks-maps of a corresponding risks-maps-sequence. The ML training dataset includes a plurality of risks-maps-sequences, generated based on a plurality of health-journeys of many different patients.

At block 1005a-1005c the Machine Learning models are trained using the training dataset. In some examples, multiple Machine Learning models are used, each model for determining correlations related to a specific type of risk-score. To this end ML-enhanced scoring module 144 can include for example, ML-enhanced severity score training module configured to generate a ML-enhanced severity scores determination model, a ML-enhanced validity score training module configured to generate a ML-enhanced validity scores determination model and a ML-enhanced interventions scores training module configured to generate a ML-enhanced interventions scores determination model.

In case of interventions, as explained above, they prescribe certain recommended changes in the patients' medical regimen, where each intervention is related to a change with respect to a certain drug or treatment. The ML analysis monitors the patient's risks-maps-sequence and can determine the health status before and after the interventions are issued and the effect the interventions have on the patient's medical condition and specifically the occurrence of medical-care events, including hospitalization and ER admissions. By applying a Machine Learning model on the training dataset which comprises many risks-maps-sequences generated from health-journeys of many patients (e.g., from hundreds of thousands to millions) accurate correlation can be obtained for different interventions.

Interventions related data (also referred to as “interventions features”) is obtained from different risks-maps along the risks-maps-sequences in the training dataset and used during training of the respective ML model. The interventions related data includes for example, interventions that were recommended for each patient, data indicating whether the interventions were applied or not, time elapsed from generation of the intervention until it was applied, changes in the API (Active Pharmaceutical Ingredient/MoA (Mechanism of Action) in the drugs related to the intervention, changes in the classification of the health-item from controlled to uncontrolled (including, balanced/unbalanced and treated/untreated) or vice versa, patients' personal medical data observed along the risks-maps-sequence such as medical measurements taken before and/or after the intervention was issued, patient's affinity groups, etc. The Machine Learning model processes the risks-map sequences in the training dataset to determine the correlation between specific interventions, possibly in combination with interventions related data and information on observed medical-care events. Information regarding medical-care events include for example, occurrence/non-occurrence of a medical care-event. In some examples, more detailed information regarding medical-care events can be used for further enhancing the accuracy of the correlation between interventions and the medical outcome. For example, information on medical-care events may also include data extracted from patient's discharge summary that is submitted following discharge from the hospital and is often available as part the patient's EMR. A discharge summary often specifies various drugs which are prescribed to the patient following discharge. By comparing these drugs to the drugs that were associated with the interventions specified in the risks-maps-sequence preceding the hospitalization event, the connection between the intervention and the hospitalization can be deduced. If for example, drug A that was prescribed to the patient (e.g., for reducing hypertension) following discharge is the same that was indicated to the patient in an “add drug” intervention (for reducing hypertension), this supports the correlation between the intervention and occurrence of the medical-care event.

Based on these correlations, the Machine Learning model is trained to provide for the interventions in the current risks-map, interventions scores that more accurately represent the respective risk of occurrence of a medical-care event that is related to these interventions.

For example, consider a risks-maps-sequence extending over a 6-month period where a personalized risks-map instance is generated for each month along the period. Assume that an intervention is determined based on the first personalized risks-map in the risks-map sequence having a respective type (e.g., replace drug). The following risks-maps along the risks-map sequence are analyzed to determine whether the intervention has been applied, and if it has, whether it positively affected respective measurements.

If the intervention was generated based on an ADR of high blood pressure caused by drug ‘A’ and includes a recommendation to replace drug ‘A’ with drug ‘B’, the risks-maps-sequences are analyzed to determine whether drug replacement resulted in controlled blood pressure and whether the patients were hospitalized or ER-admitted during the health-journey period, for reasons that are related to high blood pressure. If the analysis shows that patients that replaced the drug exhibited reduced blood pressure and reduced hospitalization rates, while patients that did not replace the drug, exhibited increased blood pressure and greater hospitalization rates, this indicates that this intervention presents high risk of hospitalization in case the intervention is not followed. If on the hand other, the health-journey analysis shows that patients that replaced the drug and patients that did not replace the drug exhibited similar hospitalization rates, this indicates that this intervention presents low risk of hospitalization.

Furthermore, the ML analysis enables to enhance the accuracy of the risk-scores by increasing their resolution. By applying the Machine Learning model on risks-maps-sequences, it is possible to classify interventions in greater detail and identify more specific correlations. Several intervention types have been described, which classify interventions according to a recommended action, which is provided for correcting a respective error and reduce potential risks, in the medication regimen. These intervention types include for example, “replace drug”, “increase/reduce dosage”, “remove drug” and “add new drug”. Machine Learning enables to expand the variety of intervention types and assign each type with an accurate ML-enhanced intervention score. The Machine Learning further enables to determine correlations between specific interventions and the observed medical outcome while considering various combinations of additional parameters such as, other drugs prescribed to the patient, other health conditions that the patient has, drug adherence, etc., and provide risk-scores that reflect the dependence between these combinations of parameters and the medical outcome.

For example, each intervention type can be classified, in addition to the action types mentioned above, also according to the specific drug (and/or the active pharmacological ingredient (API) of the drug) or treatment which is related to the intervention. Thus, a more granular classification of intervention types can be implemented. For example, an intervention of type “replace drug A” may be assigned with a different intervention score than intervention of type “replace drug B”, as each type of these two interventions may exhibit a different correlation with medical outcome such as hospitalization or ER admissions.

Another example for increasing the resolution of interventions risks scores enabled by Machine Learning analysis of the risks-maps-sequence is related to the correlation between interventions (or different combinations of interventions) and specific affinity groups. For example, age groups, place of residence, income class, or some other demographic or social determinant parameter.

Likewise, the intervention features can further include specific combinations of interventions, where the ML analysis of the risks-maps-sequence determines intervention scores based on such combinations. It can be assumed that when a patient is provided with more than one intervention, the plurality of interventions may indicate a synergetic effect on the risk rather than a simple additive effect. Accordingly, the Machine Learning model is trained to determine correlations between observed medical-care events and specific combinations of different interventions and other intervention features.

Thus, according to one example, intervention types may include a combination of features such as: action type (e.g., replace drug), drug type (API), and age group (e.g., 60-70). According to another example, intervention types may include a combination of action type (e.g., replace drug), drug type, age group (e.g., 60-70), gender, other interventions, adherence as reported by the patient, etc. According to another example, intervention types may include a combination of features such as: action type (e.g., stop drug), drug type (API), other drug types (MoA, e.g. Beta blockers) identified in the patients medication regimen, etc. The Machine Learning model is trained to determine a correlation between the combination of features and the related risk of occurrence of medical-care event and assign a respective intervention score to interventions accordingly.

Increasing the resolution of the intervention classification is accompanied by an increase in the resolution of the intervention score values, where a scoring scale with a greater resolution can be used. For example, instead of using a 1 to 5 scoring scale, the resolution of the scoring scale is increased allowing more accurate risks scores, e.g., a 1 to 100 or 1 to 1000 scoring scale.

Like the intervention risks scores, ML analysis of the risks-maps-sequence enables to enhance the validity scores and severity scores as well. With respect to validity scores, considering that each health-item may be associated with various types of activators that support the probability that the patient suffers from the health condition related to the health-item, the Machine Learning model is trained to use data obtained from many patients to determine more accurate correlations between specific activators and respective health conditions and thus to obtain more accurate activation of the respective health-items. If the Machine Learning model finds that a certain activator has a strong causative connection to a certain medical condition the respective validity score that is assigned to the activator when activating a health-item representing that medical condition may be increased and vice versa.

Activators related data (also referred to as “validity features”) is obtained from different risks-maps along the risks-maps-sequences in the training dataset and used during training of the respective ML model. Validity features include for example, health-item activators recorded with respect to risks-maps, patients' personal medical data observed along the risks-maps-sequence such as changes in measurements (activators) observed along the risks-maps-sequence, patient indexes (e.g., BMI, ADL), affinity groups, etc.

The Machine Learning model processes the risks-map sequences in the training dataset to determine the correlation between specific activators, possibly in combination with other validity features, and information on observed medical-care events. Information regarding g medical-care events include for example, occurrence/non-occurrence of a medical care-event. In some examples, more detailed information regarding medical-care events can be used for further enhancing the accuracy of the correlation between activators and the medical outcome. For example, information on medical-care events may also include the specific hospital unit in which the patient was hospitalized. For example, in case a certain activator that activate a “coronary heart diseases” health-item shows high correlation with hospitalization in cardiac care unit, the validity of the activator is increased. Based on these correlations the Machine Learning model is trained to provide to the activators in the current risks-map, validity scores that more accurately represent their respective validity.

Assuming for example, a health-item related to “falling risk” is activated in the first personalized risks-map in the sequence, based on an ADR of drug A prescribed to the patient, and that an intervention to replace the drug has been determined. The following risks-maps along the risks-map sequence are analyzed to determine whether the intervention has been applied, and whether it positively affected the falling risk, namely, whether patients have been hospitalized or admitted to the ER due to falling. If the analysis shows, for example, that there is no significant difference in frequency of falling incidents between patients who replaced the drugs and those who did not, then the validity score of this activator is reduced.

Furthermore, the Machine Learning analysis can determine reinforcement of an activator by identifying a consistent change in a health-item activator (e.g., consistent increase in a certain measurement) that is correlated with an observed increase in medical-care events resulting from the health condition associated with the health-item. On the other hand, a consistent change in a health-item activator which does not show correlation with an increase in medical-care events, indicates weak connection between the activator and the respective health condition. For example, assuming a health-item A is activated in the first personalized risks-map in the sequence, based on a certain medical measurement (e.g., blood iron levels), Machine Learning analysis identifies whether hospitalization or ER admissions related to the health condition of health-item A, is correlated (shows statistically significant relation) with the continuous deterioration in the medical measurement. In case it is, the validity score of the medical measurement as an activator of health-item A is reinforced (e.g., increased).

The ML analysis may determine assignment of validity scores to activators according to the period extending from the initial identification of the activator and the occurrence of a related medical-care events. Reverting to the example above of a health-item related to “falling risk” activated in a first personalized risks-map in the sequence, based on an ADR of a certain drug prescribed to the patient, the Machine Learning analysis can determine what percentage of the patients were hospitalized or admitted to ER for the reason of falling at different time points along the health-journey period. For example, if falling related medical-care events are recorded in more than 50% of patients after 4 months, and in more than 80% of the patients after 6 months the validity score is adapted accordingly, where different validity scores are assigned according to the time elapsed from the initial activation of the respective health-item, i.e., a greater validity score is assigned to patients after 6 months.

Furthermore, the ML analysis can provide correlation between validity scores and specific affinity groups. For example, the falling risk related to the ADR can be correlated with different age groups and/or gender and a respective validity score can be assigned to patients depending to their age group and/or gender (e.g., a higher hospitalization or ER admission related to falling are recoded with men between the ages 70-80, would grant such individuals a higher validity score for a health-item related to this ADR). Yet further the ML analysis can provide correlation between validity scores and specific affinity groups (including affinity groupings based on social determinants of health) and other health conditions indicated in the risks-maps. For example, the falling risk related to the ADR can be correlated with different age groups and/or gender and osteoporosis and a respective validity score can be assigned to patients depending to their age group and/or gender and whether they suffer from osteoporosis.

Severity related data (also referred to as “severity features”) is also obtained from different risks-maps along the risks-maps-sequences in the training dataset and used during training of the ML model. The severity features, include for example, the medical condition represented by each health-item in the risks-map.

The Machine Learning model processes the risks-map sequences in the training dataset to determine the correlation between specific health-items, and information on observed medical-care events. Information regarding medical-care events include for example, occurrence/non-occurrence of a medical care-event. In some examples, more detailed information regarding medical-care events can be uses for further enhancing the accuracy of the correlation between health-items and the medical outcome. This may include for example, information on the hospitalization period (how many days), the medical procedures that were applied on the patient during hospitalization or following discharge (e.g., surgery, therapy types, treatments, etc.), the hospital unit in which the patient was hospitalized, the type of medications that were prescribed to the patients following discharge, survival rate, etc. This information provides more accurate indication of the severity of the medical outcome and is used during the Machine Learning analysis to correlate between certain health-items (and the respective health condition they represent) and the observed medical outcome and to assign more accurate severity scores accordingly. In further examples, the severity of medical-care events can be classified according to capital spent due to the hospitalization/ER admission, where a higher health expenditure associated with the event indicates a greater severity and prescribes a greater severity score.

Based on these correlations the Machine Learning model is trained to provide to the health-items in the current risks-map, severity scores that more accurately represent the respective severity of a medical-care event that correlates to these health-items.

For example, if the Machine Learning analysis finds that a certain health-item, related to a certain health condition, has strong correlation to a complicated surgery the respective severity score may be increased. If on the other hand Machine Learning analysis finds that a certain health-item, related to a certain health condition, has strong correlation to short ER admissions, that is not accompanied by any significant change in the prescribed medications, the respective severity score may be decreased.

The ML analysis may determine assignment of severity scores according to the period extending from the initial identification of the activator and the occurrence of a related medical-care events. According to this example, the severity score assigned to different health-items is also dependent on the time lapse between activation of a health-item and the occurrence of a medical-care event (e.g., hospitalization or ER admission), where a shorter time lapse is associated with a greater severity score.

Turning to the execution phase, following the generation of a patient-specific risks-map (also referred “current risks-map”, as described above with reference to FIG. 3), the trained Machine Learning is used for determining ML-enhanced core risk-scores for health-items in the risks-maps. Notable, while the following description pertains to a single patient, the same operations can be concurrently applied for the determination of risks-scores for multiple patients.

In general, current risks-map is generated based on the most updated personal medical data and is dedicated to presenting the most updated medical status of the patient. In addition, a patient specific risks-maps-sequence is generated from a corresponding patient's health-journey recorded over a certain health-journey period (block 1009). Notably, the current risks-map can be part of the patient specific risks-maps-sequence and can be generated together with the other risks-maps in the sequence. Optionally, in some examples, as part of the generation of the risks-maps-sequence (including the current risks-map) core risk-scores values are assigned in each risks-map in the risks-maps-sequence, determined by using rule-based computer logic, as explained above. This includes validity scores, severity scores and interventions scores which are assigned in each risks-map in the risks-maps-sequence.

Risks-maps-sequences are provided as input to the trained ML-enhanced intervention score determination model for obtaining ML-enhanced intervention scores, to the trained ML-enhanced validity score determination model for obtaining ML-enhanced validity scores and to the trained ML-enhanced severity score determination model for obtaining ML-enhanced severity scores (blocks 1011a, 1011b, 1011c). Each of the ML-models operates by processing the risks-maps-sequence, identifying the respective features (intervention features, validity features and severity features, collectively referred to herein as “risk-scores features”) and providing respective ML-enhanced core risk scores to the relevant health-items in the current risks-map that reflects the correlation of the features with the occurrence of medical-care events as described above.

The trained Machine Learning models can be used as part of the risk stratification and intervention process, when generating a risks-map of a certain patient, for determining ML-enhanced core risk-scores and improving the overall accuracy of the process. In some examples, ML-enhanced core risk-scores are used instead of the core risk-scores described above. For example, ML-enhanced validity score determination model can be executed for determining ML-enhanced validity scores instead of the operations described with respect to block 411, ML-enhanced severity score determination model can be executed for determining ML-enhanced severity scores instead of the operations described with respect to block 415, and ML-enhanced intervention score determination model can be executed for determining ML-enhanced intervention scores instead of the operations described with respect to block 315.

Once the ML-enhanced core risks scores have been calculated, ML-enhanced health-items risks scores, can be calculated using the core ML-enhanced core risks scores as explained above with respect to blocks 317 and 319 in FIG. 3. A patient risk-score (also referred to as “ML-enhanced patient risk-score”) can be then calculated from the collection of ML-enhanced core risks scores calculated for a certain map. When applying the risk stratification on a group of patients (cohort), the ML-enhanced patient risk-score can be used for classifying the patients according to their respective risk instead of the patient risk-score that is calculated based on the core risk-scores.

Turning now to FIG. 11, this shows an operations flow of Machine Learning analysis for determining progressive risk-scores, according to some examples of the presently disclosed subject matter. By way of example only, operations described with respect to FIG. 10 can be executed by health-journey manager 140 and more specifically by progressive risk-scores module 145. The generation and application of progressive risk-scores can be done as part of the flow described above with respect to FIGS. 3, block 323. It is noted that although the training of the Machine Learning model and its execution is described in a single flow, this is done for ease of understanding and simplicity only. Training of the Machine Learning model and its execution can be performed asynchronously and/or by different computer devices, possible located remotely one from the other.

Earlier it was explained that patients' risks-scores are calculated based on the health-item risks scores calculated in the patient's risks-map, which are each calculated in turn based on the core risk-scores or based on the ML-enhanced core risk-scores. Here the generation of ML-enhanced progressive patient risk-score is described (referred to herein as “progressive risk-score”) which is another type of patient risk-score that is determined based on the progression of health-items and their combinations observed over a risks-maps-sequence. Given a certain health-item, the risks-maps-sequence is analyzed to identify occurrences of specific types of health-items in the different risks-maps and determine a respective sequence of the health-items along the risks-maps-sequence referred to herein as “health-item sequence”. As further discussed below the health-item sequences are used as features that characterize the risks-maps-sequence and are processed by a Machine Learning model dedicated to assigning a respective patient risk-score based on a correlation between the health-item sequences (and their specific combination) and an expected medical outcome, namely correlation with medical-care events such as hospitalization or ER admission. According to this approach, rather than using information from a single risks-map, the Machine Learning model uses health-item sequence obtained from multiple maps, thereby improving the accuracy of the health-items risk determination.

During a training phase a Machine Learning model (implementing for example regression algorithms) is trained to assign respective patient risk-scores to a specific risks-maps (e.g., current risks-map), according to the correlation between the compilation of health-item sequences observed in the respective risks-maps-sequences and the observed medical outcome. To this end, initially a ML training dataset is generated. Operations related to block 1101-1103 describing the generation of a ML training dataset were described above with respect to blocks 1001-1003 in FIG. 10. The ML training dataset includes a plurality of risks-maps-sequences, each including a plurality of risks-maps generated with respect to a corresponding time point along a health-journey period and including information on the medical-care events recorded for different patient. At block 1105 the Machine Learning model is applied on the training dataset to thereby obtain the trained machine model.

As noted above, although the training of the Machine Learning model and its execution is described in a single flow, this is done for ease of understanding and simplicity only. The training of the Machine Learning model and its execution can be performed asynchronously and/or by different computer devices, possible located remotely one from the other.

Health-item sequences are identified and processed to determine correlations between specific health-item sequences and different combinations of health-item sequence with observed medical-care events. Several examples are described herein below. One example is related to the relation between the number of consecutive health-items of a certain type, which are observed in the health-item sequences and their correlation with hospitalization and/or ER admissions. The Machine Learning analysis may find with respect to a first health-item sequence that appearance of a health-item in 2 consecutive risks-maps along the risks-maps-sequence has a 30% correlation with hospitalization and/or ER admission, while appearance of the health-item in 4 consecutive risks-maps along the risks-maps-sequence has a 90% correlation with hospitalization and/or ER admission. The Machine Learning analysis may find with respect to a second health-item sequence that appearance of the health-item in 2 consecutive risks-maps along the risks-maps-sequence indicates a 10% correlation with hospitalization and/or ER admission while appearance of the health-item in 4 consecutive risks-maps along the risks-maps-sequence indicates a 50% correlation with hospitalization and/or ER admission.

In another example, the Machine Learning model identifies correlation between appearance of a combination of specific health-items and medical-care events. For example, given a first health-item sequence of a first health-item and a second health-item sequence of a second health-item, the correlation between the two sequences and the occurrence of medical-care events is determined. Correlations may depend on the types of health-items and their specific progression features along the risks-maps-sequence.

For instance, Machine Learning analysis may find that appearance of a first health-item of a certain health category (e.g., heart problems) in several consecutive risks-maps (say 2 risks-maps) followed by appearance of a second health-item from the same category in the following risks-maps, indicates an increase of 50% correlation with hospitalization and/or ER admission as compared to appearance of the first health-item alone. This correlation may be further analyzed to determine more specific variations, for example, the difference in correlation according to the number of consecutive risks-maps that exhibited overlapping appearances of the two health-items.

Another example is related to information indicating whether the health-item is classified as controlled or uncontrolled in each risks-map along the risks-maps-sequence. For example, a first health-item sequence may indicate that appearance of health-item A in 3 consecutive risks-maps along the risks-maps-sequence indicates a 90% correlation with hospitalization and/or ER admission in case the health-item is uncontrolled, and 30% correlation with hospitalization and/or ER admission in case the health-item is controlled.

The Machine Learning model applied on the training dataset is configured to identify correlations of different combinations of different features with the occurrence of medical-care events and determine a patient's risk-score scoring model which reflects these correlations and can provide patient's risks-scores accordingly.

Turning to the execution phase, given a certain risks-map and a respective risks-map-sequence, the trained Machine Learning model is configured to determine a respective patient risk-score. Machine Learning. A patient specific risks-maps-sequence is generated from the respective patient's health-journey (block 1107). If a current risks-map is not available, it can be generated as part of the risks-map-sequence. The risks-maps-sequence comprises a corresponding health-item progression for each health-item that appears in the risks-maps. According to some examples, such health-items sequences are identified in the risks-maps-sequence (block 1109). The Machine Learning model (progressive risk-scores ML-execution model) is used for processing the health-item sequences and providing a respective progressive risks-score of the patient as output according to the specific combination of features that were identified. As explained above, the risks stratification and intervention process can be applied on a large population (cohort) of patients for the purpose of prioritizing the patients according to their respective overall risk of occurrence of medical-care event. In some examples, the progressive risk-score is used together with the ML enhanced patient risk-score, e.g., by aggregating the two scores into a single value (“combined patient risk-score”). When applying the combined patient risk-score on a large population of patients more accurate differentiation and prioritization of patients according to their risk to experience a medical-care event is obtained.

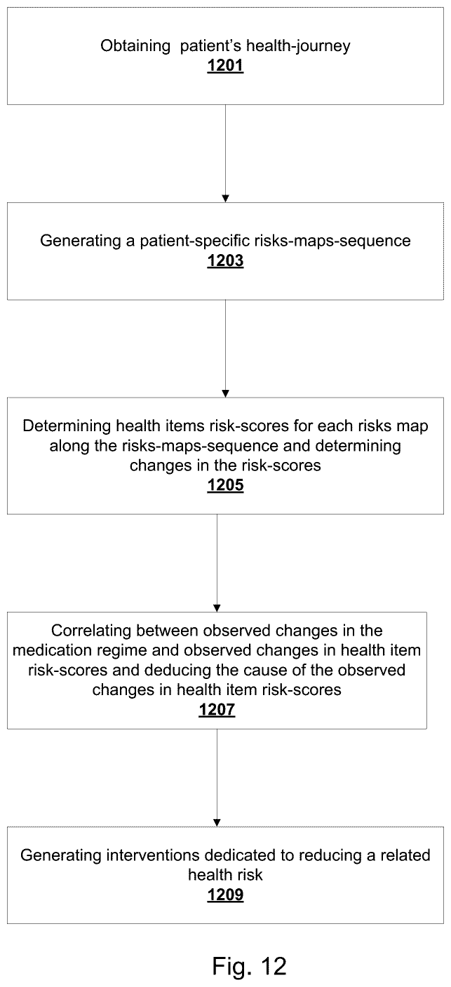

Turning to FIG. 12 this shows an operations flow executed during risk-stratification and intervention process using health-journey data, according to some examples of the presently disclosed subject matter. Previously it was explained that interventions are generated by applying computer logic and processing a current risks-map generated with respect to a certain time instance. Interventions are generated for risk-related health-items identified in the current risks-map based on the information available from the risks-map indicating their cause of activation. For example, a health-item that is activated due to an ADR of a certain medication may induce a “replace-drug” intervention.

However, in some cases it may be difficult to accurately associate between a certain medical condition and the specific cause that created this condition. This task is especially challenging in patients that suffer from multiple health problems (comorbidities) and are prescribed with multiple medications for treating these problems (polypharmacy). Thus, according to further examples of the presently disclosed subject matter, data obtained from a patient's health-journey rather than from only a single risks-map is obtained and used in the intervention generation process. Operations described with respect to FIG. 12 can be performed for example, by health-journey manager 140 and more specifically by health-journey interventions analysis module 146. Operations described with reference to FIG. 12 can be executed for example as part of 305 in FIG. 3.

At block 1201 a patient-specific risks-maps-sequence is generated from the patient's health-journey (or otherwise obtained) along with a current risks-map, as described above with reference to FIG. 10. As part of the generation of the risks-maps intervention may be generated as described above with reference to block 305.

At block 1203 health-journey is processed to determine changes in the medication regimen of the patient, which occurred along the health-journey up to (including) the current risks-map. These changes include for example, changes in medications and other treatments, including addition of new drugs or other treatments (e.g., dialysis) or removal of previously prescribed drugs or other treatments, changes in dosage and/or frequency of intake of existing medications, changes in patient's adherence to prescribed drugs, etc. According to some examples, the changes are determined by comparing between personal medical data which is associated and used during the generation of different personalized risks-maps along the risks-maps-sequence. According to some examples, each time an updated risks-map is generated, new personal medical data is compared with the past personal medical data and any changes in the medication regimen are identified and recorded (e.g., in data-repository 129).

Details on any observed changes in the medication regimen are obtained and processed. Such details include, for example, the date of change in the medication regimen. This may include, for example, any one of: the date the patient has started to take a new medication, the date the patient has stopped taking an existing medication, the date the dosage or frequency of an existing medication has changed, etc. This information can be retrieved, for example, from the patient's EMR.

Since many drugs do not have an immediate effect on the patient after their initial consumption, in some examples, information on changes in the patient's medication regimen may further include information pertaining to the delayed onset of drugs effects of specific newly prescribed drugs. This information can be obtained for example from the medical association database 105. Clearly in some cases the drug effect (or effect of some other treatment) is immediate and in such cases the delayed onset may be set to zero. An estimated drug effect onset date can be calculated based on the date of change made with respect to a certain drug as obtained from the personal medical data and the corresponding information related to the delayed onset of the drug effect.

At block 1205, health-item risk-scores are calculated for each map along the risks-maps-sequence and a sequence of health-item risks scores is obtained indicating changes in the health-items risks scores along the risks-maps-sequence. The process of calculating health-items risks score is explained above. The respective dates of the observed changes in the risk-scores can be estimated for example, based on the date on which these changes were detected, i.e., the date of a corresponding risks-map which exhibits the change.

At block 1207 observed changes in the patient's medication regimen are correlated with changes in the health-item risks scores observed along the risks-maps-sequence. Changes in specific medication/treatments are also matched to respective changes in health-item risks scores observed in the currently updated personalized risks-map taking into consideration their respective estimated drug effect onset date and the respective estimated date of occurrence of the change in risk. A change in medication/treatment that has matching dates with a certain change in a health-item risk is identified as a possible cause for the observed change in risk.

Thus, for example if a certain health-item exhibits an increase in its respective risks score and this increase is found to be correlated with a change in the medication regimen such as, commencement of taking a new drug, or cessation of taking a drug, the observed change is identified as a possible cause of the change in health-item risks score.

At block 1209 interventions dedicated to reducing or eliminating the risk associated with certain health-items are generated (or refined if they already exist) based on the deduced causes of these risks.

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present presently disclosed subject matter.

1. A computer implemented method of automatic stratification of a group of patients according to risk of occurrence of a medical-care event, the method comprising:
for each patient, in the group of patients:
processing a patient's health-journey that comprises historical personal medical data of the patient collected over a past period and generating a risks-maps-sequence comprising a plurality of risks-maps, each risks-map in the risks-maps-sequence is generated based on personal medical data which was available at a certain time point along the past period;
wherein the risks-maps-sequence comprises a current risks-map; wherein each risks-map is a data-structure comprising: i) a plurality of relevant health-items, each relevant health-item is a data object that represents a respective health condition which is identified as relevant to a medical status of the patient by at least one activator that includes medical data; and ii) at least one intervention that includes data that prescribes a change to a drug or a treatment that has been identified in the risks-map to be related to a respective health-risk to the patient;
providing the risks-maps-sequence as input to one or more Machine-Learning (ML) models, dedicated to determining a plurality of risk-scores for the current risks-map;
determining, based on the plurality of risk-scores assigned to the current risks-map, a patient's risk-score, that indicates a relative risk of occurrence of a medical-care event to the patient;
and classifying patients in the group of patients according to their respective patient's risk-score.

2. The method of claim 1 wherein the plurality of risk-scores include:
a respective validity score to one or more activators of a relevant health-item in the current risks-map, which indicate collectively a level of certainty that the respective health conditions is relevant to the patient;
a respective severity score to each relevant health-item in the current risks-map, which indicates a severity of a health risk that is related to the respective health condition; and
a respective intervention score to each intervention in the current risks-map, that indicates a correlation between the intervention and occurrence of a medical care event;

3. The method of any one of claims 1 and 2, wherein the intervention is related to a respective relevant health-item in the current risks-map, which represents a health condition that is related to the respective health-risk to the patient, the method further comprising, determining for each relevant health-item in the current risks-map, based on the plurality of risk-scores, a respective health-item risk-score and determining the patient's risk-score based on a combination of the plurality of risk-scores in the current risks-map.

4. The method of any one of the preceding claims further comprising:
for each patient in the group:
providing the risks-maps-sequence as input to a Machine Learning (ML) model dedicated to determining a respective progressive risk-score of the patient that indicates a correlation between a combination of one or more health-item sequences found in the risks-maps-sequence and a risk of occurrence of a medical-care event to the patient;
determining a combined patient's risk-score based on the patient's risk-score and the progressive risk-score; and
classifying patients in the group of patients according to their respective combined risk-scores.

5. The method of any one of the preceding claims further comprising, during generating the current risks-map for each patient:
processing the patient's health-journey and identifying at least one dynamic activator; wherein a dynamic activator comprises a sequence of medical data values of a certain type recorded in the patient's health-journey, wherein each medical data value in the sequence is recorded at a different time along a period of the health-journey and the sequence of medical data values is characterized by a distinctive pattern;
determining a certain health condition that exists in correlation with the dynamic activator; and
classifying a health-item representing the certain health condition as a relevant health-item based on the correlation.

6. The method of claim 5 further comprising:
identifying in the patient's health-journey, additional medical data, other than the dynamic activator;
determining a health condition that exists in correlation with the dynamic activator and the additional medical data; and
classifying a health-item representing the certain health condition as a relevant health-item based on the correlation and the additional medical data.

7. The method of claim 5, wherein a plurality of dynamic activators that comprise the same type of medical data are characterized each by a different distinctive pattern, and is each correlated with a different health condition of a plurality of health conditions; the method further comprising:
determining respective features charactering the distinctive pattern;
determining based on the respective features a correlation between the dynamic activator and a specific health condition out of the plurality of health conditions; and
identifying a health-item representing the specific health condition as a relevant health-item.

8. The method of any one of the preceding claims further comprising determining the at least one intervention comprising:
identifying in the relevant health-items, one or more risk-related health-items that are each indicative of a health risk related to a drug prescribed to the patient, comprising:
obtaining from the personal medical data of the patient, information about administration of a drug or treatment for treating a respective health condition;
comparing the information with data indicating recommended administration of the drug or treatment; classifying the respective relevant health-item representing the health condition as risk-related in case a discrepancy is found between the information and the recommended administration; and generating an intervention dedicated to correcting the discrepancy.

9. The method of any one of the preceding claims further comprising determining the at least one intervention comprising:
identifying in the relevant health-items, one or more risk-related health-items that are each indicative of a health risk related to a drug prescribed to the patient, comprising:
obtaining from the personal medical data of the patient, information about a drug that is being administered for treating a health condition and that is related to a health risk which may be caused from usage of the drug; and generating an intervention dedicated to alleviating the health risk.

10. The method of any one of the preceding claims, wherein the medical-care event includes any one of hospitalization and ER admission.

11. The method of claim 3, further comprising determining the at least one intervention comprising:
determining for each relevant health-item in each risks-map in the risks-maps-sequence a respective health-item risk-score;
identifying one or more changes in a drug or treatment in a medication regimen observed along the patients' health-journey;
identifying one or more changes in health-item risk-scores of respective relevant health-items observed along the risks-maps-sequence;
identifying a correlation between a time of a change in a drug or treatment and a time of a change in a health-item risk-score of a respective relevant health-item;
deducing based on the correlation that the change in the drug or treatment caused the change of the health-item risk-score of the respective relevant health-item;
generating, according to the deducing, an intervention dedicated to alleviating a health risk related to the respective drug or treatment.

12. The method of claim 11, wherein the at least one change in the respective drug or treatment is commencement of taking a new drug, the method further comprising obtaining data indicative of a respective drug delay onset period and adapting the time of the change according to the respective drug delay onset period.

13. The method of any one of the preceding claims, wherein the one or more ML models includes:
a first ML model dedicated to assigning validity scores based on combinations of validity features identified in the risks-maps-sequence;
a second ML model dedicated to assigning severity scores based on combinations of severity features identified in the risks-maps-sequence; and
a third ML model dedicated to assigning interventions scores based on combinations of interventions features identified in the risks-maps-sequence.

14. The method of any one of the preceding claims, wherein the current risks-map is generated based on most updated personal medical data in the patient's health-journey.

15. A computer system comprising a processing circuitry comprising at least one processer and computer memory, the processing circuitry is configured to execute a method of automatic stratification of a group of patients according to risk of occurrence of a medical-care event, as described in any one of claims 1 to 14.

16. A computer product operable in a computer and recorded on a non-transitory computer-readable medium for automatic stratification of a group of patients according to risk of occurrence of a medical-care event, wherein the product is produced by the processes as described in any one of claims 1 to 14.

17. A non-transitory computer readable storage medium tangibly embodying a program of instructions that, when executed by a computer, cause the computer to perform a method of automatic stratification of a group of patients according to risk of occurrence of a medical-care event as described in any one of claims 1 to 14.

18. A computer product operable in a computer and recorded on a non-transitory computer-readable medium for automatic stratification of a group of patients according to risk of occurrence of a medical-care event, wherein the product is produced by the processes of:
for each patient, in the group of patients:
processing a patient's health-journey that comprises historical personal medical data of the patient collected over a past period and generating a risks-maps-sequence comprising a plurality of risks-maps, each risks-map in the risks-maps-sequence is generated based on personal medical data which was available at a certain time point along the past period;
wherein the risks-maps-sequence comprises a current risks-map; wherein each risks-map is a data-structure comprising: i) a plurality of relevant health-items, each relevant health-item is a data object that represents a respective health condition which is identified as relevant to a medical status of the patient by at least one activator that includes medical data; and ii) at least one intervention that includes data that prescribes a change to a drug or a treatment that has been identified in the risks-map to be related to a respective health-risk to the patient;
providing the risks-maps-sequence as input to one or more Machine-Learning (ML) models, dedicated to determining a plurality of risk-scores for the current risks-map, that include:
a respective validity score to one or more activators of a relevant health-item in the current risks-map, which indicate collectively a level of certainty that the respective health condition is relevant to the patient;
a respective severity score to each relevant health-item in the current risks-map, which indicates a severity of a health risk that is related to the respective health condition; and
a respective intervention score to each intervention in the current risks-map, that indicates a correlation between the intervention and occurrence of a medical care event;
determining, based on the plurality of risk-scores assigned to the current risks-map, a patient's risk-score, that indicates a relative risk of occurrence of a medical-care event to the patient;
classifying patients in the group of patients according to their respective patient's risk-score.

19. A computer implemented method of automatic stratification of health risks in a medication regimen administered to a patient, the method comprising:
generating a risks-map data-structure comprising:
selectively adding to the data-structure a plurality of relevant health-items, each relevant health-item is a data object that represents a respective health condition which is identified as relevant to a medical status of the patient by at least one activator that includes medical data;
processing a patient's health-journey that comprises historical personal medical data of the patient collected over a past period and identifying at least one dynamic activator; wherein a dynamic activator comprises a sequence of medical data values of a certain type recorded in the patient's health-journey, wherein each medical data value in the sequence is recorded at a different time along a period of the health-journey and the sequence of medical data values is characterized by a distinctive pattern;
determining a certain health condition that exists in correlation with the dynamic activator; and
classifying a health-item representing the certain health condition as a relevant health-item based on the correlation.

20. The method of claim 19 further comprising:
identifying in the patient's health-journey, additional medical data, other than the dynamic activator;
determining a health condition that exists in correlation with the dynamic activator and the additional medical data; and
classifying a health-item representing the certain health condition as a relevant health-item based on the correlation and the additional medical data.

21. The method of any one of claims 19 and 20, wherein a plurality of dynamic activators that comprise the same type of medical data are characterized each by a different distinctive pattern, and is each correlated with a different health condition of a plurality of health conditions; the method further comprising:
determining respective features charactering the distinctive pattern;
determining based on the respective features a correlation between the dynamic activator and a specific health condition out of the plurality of health conditions; and
identifying a health-item representing the specific health condition as a relevant health-item.

22. The method of any one of claims 19 to 21, further comprising:
determining at least one relevant health-item in the risks-map that is indicative of a health risk related to a drug or treatment prescribed to the patient and at least one respective intervention that prescribes a change to the drug or a treatment dedicated to alleviating the health risk;
determining the at least one respective intervention comprising:
processing the patient's health-journey and generating a respective risks-maps-sequence comprising a plurality of risks-maps, each risks-map in the risks-maps-sequence is generated based on personal medical data which was available at a certain time point along the past period;
determining for each relevant health-item in each risks-map in the risks-maps-sequence a respective health-item risk-score;
generating at least one intervention that prescribes a change to a drug or a treatment that has been identified in the risks-map to be related to a health-risk to the patient, comprising:
identifying one or more changes in a respective drug or treatment in the medication regimen observed along the patient's health-journey;
identifying one or more changes in health-item risk-scores of respective relevant health-items observed along the risks-maps-sequence;
identifying a correlation between a time of at least one change in a respective drug or treatment and a time of at least one change in health-item risk-score;
deducing based on the correlation that the at least one change in the drug or treatment caused the change of the health-item risk-score of the at least one relevant health-item;
generating, according to the deducing, the at least one respective intervention dedicated to alleviating a health risk related to the respective drug or treatment.

23. The method of any one of claims 19 to 22, further comprising:
assigning to each relevant health-item in the risks-map a respective validity score based on to the at least one activator, which indicates level of certainty that the respective health conditions is relevant to the patient;
assigning to each relevant health-item in the risks-map a respective severity score which indicates a severity of a health risk that is related to the respective health condition;
assigning to each intervention in the risks-map a respective intervention score;
assigning to each relevant health-item a respective health-item risk-score calculated based on the respective validity score, the respective severity score and one or more respective intervention scores related to the relevant health-item; wherein the respective health-item risk-score is indicative of a relative risk of occurrence of a medical-care event as a result of the respective health condition.

24. The method of claim 23, wherein the medical-care event is hospitalization of the patient.

25. A computer system comprising a processing circuitry that comprises at least one processer and computer memory, the processing circuitry is configured to execute a method of automatic stratification of health risks in a medication regimen administered to a patient, as described in any one of claims 19 to 24.

26. A computer product operable in a computer and recorded on a non-transitory computer-readable medium for automatic stratification of health risks in a medication regimen administered to a patient, wherein the product is produced by the processes as described in any one of claims 19 to 24.

27. A non-transitory computer readable storage medium tangibly embodying a program of instructions that, when executed by a computer, cause the computer to perform a method of automatic stratification of health risks in a medication regimen administered to a patient as described in any one of claims 19 to 24.

28. A computer implemented method of automatic classification of health risks in a medication regimen administered to a patient, the method comprising:
generating a risks-map data-structure comprising:
selectively adding to the data-structure a plurality of relevant health-items, each relevant health-item is a data object that represents a respective health condition which is identified as relevant to a medical status of the patient by at least one activator that includes medical data;
wherein the risks-map is a data-structure comprising: i) a plurality of relevant health-items, each relevant health-item is a data object that represents a respective health condition which is identified as relevant to a medical status of the patient by at least one activator that includes medical data; and ii) at least one intervention that includes data that prescribes a change to a drug or a treatment that has been identified in the risks-map to be related to a respective health-risk to the patient;
determining the at least one intervention comprising:
processing a patient's health-journey that comprises historical personal medical data of the patient collected over a past period and generating a respective risks-maps-sequence comprising a plurality of risks-maps, each risks-map in the risks-maps-sequence is generated based on personal medical data which was available at a certain time point along the past period;
determining for each relevant health-item in each risks-map in the risks-maps-sequence a respective health-item risk-score;
generating at least one intervention that prescribes a change to a drug or a treatment that has been identified in the risks-map to be related to a health-risk to the patient, comprising:
identifying one or more changes in a respective drug or treatment in the medication regimen observed along the patient's health-journey;
identifying one or more changes in health-item risk-scores of respective relevant health-items observed along the risks-maps-sequence;
identifying a correlation between a time of a change in a respective drug or treatment and a time of a change in health-item risk-score of a respective relevant health-item;
deducing based on the correlation that the change in the drug or treatment caused the change of the health-item risk-score of the respective relevant health-item;
generating the at least one intervention dedicated to alleviating a health risk related to the respective drug or treatment.

29. The method of claim 28, wherein the at least one change in the medication regimen is commencement of taking a new drug, the method further comprising obtaining data indicative of a respective drug delay onset period and adapting the time of the at least one change according to the respective drug delay onset period.

30. The method of any one of claims 28 and 29, wherein determining a respective health-item risk-score, comprises:
assigning to each relevant health-item in the risks-map a respective validity score based on to the at least one activator, which indicates level of certainty that the respective health conditions is relevant to the patient;
assigning to each relevant health-item in the risks-map a respective severity score which indicates a severity of a health risk that is related to the respective health condition;
assigning to each intervention in the risks-map a respective intervention scores;
assigning to each relevant health-item a respective health-item risk-score calculated based on the respective validity score, the respective severity score and one or more respective intervention scores related to the relevant health-item; wherein the respective health-item risk-score is indicative of a relative risk of occurrence of a medical-care event as a result of the respective health condition.

31. A computer system comprising a processing circuitry that comprising at least one processer and computer memory, the processing circuitry is configured to execute a method as described in any one of claims 28 to 30.

32. A computer product operable in a computer and recorded on a non-transitory computer-readable medium for automatic stratification of health risks in a medication regimen administered to a patient, wherein the product is produced by the processes as described in any one of claims 28 to 30.

33. A non-transitory computer readable storage medium tangibly embodying a program of instructions that, when executed by a computer, cause the computer to perform a method as described in any one of claims 28 to 30.
